(19) United States
(12) Patent Application Publication
(10) Pub. No.: US 2025/0154476 A1
(43) Pub. Date: May 15, 2025

(54) IMPROVED HEPARAN SULFATE AND METHODS OF MAKING THE SAME

(71) Applicant: University of Copenhagen, Køvenhavn K (DK)

(72) Inventors: Rebecca Louise Miller, Køvenhavn (DK); Richard Karlsson, Køvenhavn (DK); Zhang Yang, Køvenhavn (DK); Jeremy E. Turnbull, Køvenhavn (DK); Henrik Clausen, Køvenhavn (DK)

(21) Appl. No.: 18/721,990

(22) PCT Filed: Dec. 21, 2022
(86) PCT No.: PCT/EP2022/087261

§ 371 Date: Jun. 20, 2024

(30) Foreign Application Priority Data
Dec. 21, 2021 (EP) 21216662.3

(51) Int. Cl. C12N 9/10; A61K 31/727

(52) U.S. Cl. CPC C12N 9/13; A61K 31/727; C12Y 208/02023

(57) ABSTRACT

According to the invention there is provided methods for making heparan sulfate with increased anticoagulant activity wherein the resulting product has a lower heterogeneity and presents an improved safety profile compared to conventional animal-sourced heparin.

### FIELD OF THE INVENTION

The present invention relates to design and production of improved heparan sulfate with potent anticoagulant activity and an improved safety profile. Currently, heparin is derived from pig intestines, and is highly heterogenous, with problems concerning drug product consistency and purity. Methods to produce heparin without the use of animals are sought after, and heparin with higher purity and better safety profile is desirable.

Platelet factor 4 (PF4) can bind heparin and the binding of PF4 to administered heparin can result in heparin-induced thrombocytopenia, a well-known adverse side effect of heparin. The present invention relates to methods to produce heparan sulfate with lower heterogeneity and with an improved safety profile. In particular, the present invention relates to heparan sulfate preparations with high anticoagulant activity and with low binding to PF4.

### BACKGROUND OF THE INVENTION

### Heparin/Heparan Sulfate

The heparin/heparan sulfate (HS) family of polysaccharides found throughout metazoan lifeforms are the most anionic polysaccharides in nature ranging from 20-200 monosaccharide units in length, and HS is ubiquitously expressed on cell surfaces and in the extracellular matrix of mammals¹. The degree and patterns of their sulfation represent huge diversity for informational cues to direct and tightly regulate biological functions. They achieve this through selective interactions with protein partners via divergent sulfated binding motifs that bind to cognate protein binding sites. Heparin/HS is produced by a complex biosynthetic machinery that initially creates a repeating disaccharide unit of uronic acid (UA) and N-acetylglucosamine (GlcNAc), where the uronic acid is either iduronic (IdoA) or glucuronic (GlcA) acid (FIG. 1). The glucosamines can be modified with an N-sulfate (NS) or remain as an N-acetyl (NAc) moiety (via action of N-deacetylase/N-sulfotransferases 1-4 (NDST1-4)). Uronic acids can subsequently be modified with an O-sulfate at the carbon-2 position by a 2-O-sulfotransferase (HS2ST1). Further O-sulfates can be added to glucosamine residues at the carbon-6 position (via 6-O-sulfotransferases 1-3 (HS6ST1-3)) and more rarely at the carbon-3 position (via 3-O-sulfotransferases 1-6 (HS3ST1-6)). Divergent patterns of sulfation created by the orchestration of these enzyme families are the key hallmarks of functionally specific protein binding sites in heparin/HS. The expression patterns of these sulfotransferases and especially the HS3STs is spatio-temporally regulated in a broad range of tissues²⁻⁴. HS3ST1 and HS3ST5 are considered to be the two main isoenzymes involved in 3-O-sulfation of the anticoagulant drug heparin^{5,6}.

### Pharmaceutical Heparin

Heparin, a member of the HS family, is a widely used anticoagulant and is the world's most sold biopharmaceutical by weight, yet it remains a poorly characterized heterogeneous animal-sourced product⁸. Heparin is produced in mast cells and unfractionated heparin (UFH) is derived from animal tissues. Most UFH is purified from porcine intestinal mucosa⁹, with low molecular weight heparins (LMWHs) being fractionated from UFH. The supply and quality of heparins are causes for concern due to infection outbreaks in animal stocks, such as the ongoing swine flu in China, and the contamination of crude heparin with over-sulfated glycosaminoglycans (GAGs) in 2007 that resulted in many deaths¹⁰.

### Anticoagulant Activity of Heparin

The mechanism of heparin's anticoagulant activity involves predominantly binding and activation of antithrombin III (ATIII), which is then able to complex and inactivate thrombin, factor Xa (FXa) and other proteases¹¹. High affinity binding of Heparin to ATIII involves a specific pentasaccharide sequence (GlcNS6S-GlcA-GlcNS3S6S-IdoA2S-GlcNS6S), whereas the interaction of ATIII and thrombin requires heparin chains of at least 18 monosaccharide units in length¹². In contrast FXa activity via ATIII activation requires only the pentasaccharide sequence and a synthetic heparin mimetic (fondaparinux), has been created based on this structure¹³. Removal of the 3-O-sulfate group on the 3-O-sulfated glucosamine (GlcNS3S6S) within the pentasaccharide sequence was shown to result in limited ATIII activity¹⁴, demonstrating the essential requirement for 3-O-sulfation for potent anticoagulant activity.

There are seven 3-O-sulfotransferases in mammals and based on genetic studies in mice, Hs3st1 is responsible for the overwhelming majority of antithrombin-binding structures^{3, 15}. HS3ST1 is thought to be essential for generating antithrombin binding sites¹⁶. This is supported by the finding that Hs3st1^{-/-} mice with deficiency in Hs3st1 have drastically reduced anticoagulant activity of their levels of AT-type HS supporting that HS3ST1 is the main 3-O-sulfotransferase responsible for biosynthesis of AT-type heparin^{3, 15}. The Hs3st1^{-/-} mice did not exhibit thrombotic phenotype and hence other 3-O-sulfotransferases, notably Hs3st5, may create sufficient AT-type HS to compensate for loss of Hs3st1. HS3ST1 and HS3ST5 form a homologous subgroup, sharing 71 percent identity in the sulfotransferase domain, indicating that these share kinetic properties and functions⁶. Therefore, these two sulfotransferases have in common the capacity to generate a binding site for antithrombin and thus are designated AT-type sulfotransferases.

### Heparin-Induced Thrombocytopenia

Major complications of heparin and LMWH in clinical use include both bleeding and thrombosis. The structural heterogeneity of heparins provides the avidity to complex with large numbers of proteins including plasma proteins, which can lead to adverse consequences of unpredictable anticoagulation and also life-threatening heparin-induced thrombocytopenia (HIT)¹⁷. HIT can be non-immune or immune-mediated, both resulting in decreased platelet counts. Platelets produce a protein called platelet factor 4 (PF4; also called CXCL4), which is capable of forming large heparin-PF4 complexes; in immune HIT antibodies to these complexes are induced and platelets are activated, resulting in the formation of blood clots and low platelet levels^{17, 18}. Heparin has the highest incidence of HIT at around 5% of patients, whereas LMWH has an incidence of around 1%¹⁹. Heparin/LMWH binding to PF4 has previously been demonstrated to require N-sulfation of the glucosamine (GlcNS) and 2-O-sulfation of the uronic acid (UA2S)²⁰.

### Genetic Engineering of Mammalian Cells for Production of Heparin

Heparin remains one of few pharmaceuticals still isolated from animal tissues without thorough structural characterization⁸. Production of heparin in mammalian cells is considered a potential alternative to current animal sources, and advances have been made through overexpression and directed KI of enzymes functioning in the HS biosynthetic pathway²¹. Chinese hamster ovary (CHO) cells have historically been chosen for genetic engineering²², and initial efforts to systematically engineer GAG biosynthetic pathways have used genetic engineering for generating large libraries of individual cells that display different repertoires of HS, chondroitin sulfate (CS) and dermatan sulfate (DS) structures²³.

WO 2017/106782 A1 and WO 2018/112434 A1 are patent publications that relate to glycosaminoglycans derived from genetically modified cells, wherein the cells are made transgenic and/or deficient for a large number of enzymes in the GAG biosynthetic pathways. However, there is no disclosure or suggestion of the use of HS3ST4 to increase the anticoagulant activity of heparan sulfate or heparin.

As stated in the Uniprot database entry on human HS3ST4 (www.uniprot.org/uniprot/Q9Y661) as accessed on 15 Dec. 2021, unlike HS3ST1, which is responsible for converting non-anticoagulant heparan sulfate to anticoagulant heparan sulfate, HS3ST4 is believed not to convert non-anticoagulant heparan sulfate into anticoagulant heparan sulfate.

### SUMMARY OF THE INVENTION

Unexpectedly, it has been found that treating heparan sulfate with HS3ST4 increases the anticoagulant activity of HS.

The present invention exploits these findings, namely that heparan sulfate produced with HS3ST4 as opposed to other HS3ST isoenzymes (HS3ST1, 2, 3A, 3B, 5, and/or 6) is improved unexpectedly.

HS3ST4 produced heparan sulfate has anticoagulant activity and no or low binding to PF4. This alleviates induction of the adverse side effect of Heparin-induced thrombocytopenia (HIT) commonly seen in patients receiving animal-derived heparin.

There are described below various aspects of the invention involving genetic engineering of heparin/HS biosynthesis in mammalian cell lines using a specific combination of isoenzymes including the 3-O-sulfotransferase HS3ST4. The present invention further relates to chemoenzymatic synthesis of heparan sulfate with anticoagulant activity and with no or weak affinity for binding to PF4 by using HS3ST4. The heparan sulfate produced according to the methods of the invention provides a safer alternative to known animal-derived heparin.

### BRIEF DESCRIPTION OF THE DRAWINGS

FIG. 1 illustrates the functions of enzymes involved in the cellular biosynthesis of heparin/HS. The repeating disaccharide units of heparin/HS are linked to serine residues in proteins by a tetrasaccharide linker consisting of xylose-galactose-galactose-glucuronic acid. The N-acetylglucosamine residues of the disaccharide repeats can be deacetylated and sulfated on the N-position and further sulfated on the 3-0 and 6-0 positions. The glucuronic acids can be epimerized to iduronic acids and sulfated on the 2-0 position.

FIG. 2 shows a dendrogram generated by multiple sequence alignment (ClustalW) of the full coding sequences of the seven human HS3STs based on amino acid sequence (left part), and a graphical depiction of the main structural features of the HS3ST enzymes including the catalytic domain in the C-terminal region (approximately 250 amino acids) (right part). HS3ST1 has a cleavable signal peptide and is predicted to be a soluble secreted enzyme, while all others have typical type II membrane domain sequences close to their N-terminal ends. All HS3STs carry N-glycans, where one glycosite is conserved between all isoenzymes, and one additional site is conserved only between HS3ST2, 4, 3A and 3B. Designations used: SIG—Signal peptide sequence, TM—Type II transmembrane domain sequence, N-glycan sites are indicated with N-glycan symbols above the glycosylation site.

FIG. 3 illustrates the genetic engineering of CHO cells to individually express the human HS3STs. CS/DS biosynthesis was ablated by knock-out (KO) of Chondroitin sulfate N-acetylgalactosaminyltransferase-1 (CSGalNAcT1) and -2, (CSGalNAcT2) and Chondroitin sulfate synthase 1 (Chsy1), followed by individual stable targeted knock-in of all 7 of the human HS3STs.

FIG. 4 shows the verification of HS3ST expression in CHO cells. (A) Immunocytochemistry analysis of the primary selected CHO HS3ST knock-in (KI) clones using antibodies probing the V5- or S-tags c-terminally fused to the enzymes for enabling verification of cellular enzyme expression. (B) SDS-PAGE Western blot analysis against the V5- or S-tagged HS3ST enzymes from the primary selected CHO HS3ST KI clones, comparing enzyme levels in cell lysate and media. Expected molecular weight below the images is based on amino acid sequence and predicted presence of N-glycans.

FIG. 5 shows Western blot analysis of cell lysates of the complete set of genetically engineered CHO HS3ST KI clones using antibodies against the c-terminally added V5- and S-tags. The primary selected KI clones are indicated in bold. β-actin was used as loading control.

FIG. 6 illustrates disaccharide analysis of HS from CHO cells individually expressing the human HS3STs and the parental cell line with knock out of CSGalNAct1/CSGalNAct2/Chsy1 (designated CHO KO CS). Heparinase digested HS was analyzed by C18 HPLC and compared against 20 pmol disaccharide standards. Quantification of disaccharides are presented as a relative percentage of all disaccharides identified for each sample.

FIG. 7 illustrates ATIII and PF4 binding to heparin/HS. To generate a cell-based anticoagulant, functional assays for anticoagulant activity such as the anti-factor Xa (FXa) assay are used for comparing HS from genetically engineered cells with clinical heparin/LMWHs. A major side effect of heparin is HIT due to PF4 binding to heparin chains; therefore, PF4 binding of HS/heparin/LMWHs is measured to identify low-binding variants that would not generate this side effect.

FIG. 8 shows anticoagulant activity and PF4 binding of HS from genetically engineered CHO cells and clinically used heparins. (A) Dose response data from FXa assay determining the anticoagulant activity of HS from genetically engineered CHO cells, compared to clinical heparin/LMWHs. The key under the radar chart shows the quantity of HS/heparin used for the experiment. Absorbance at 405 nm is indicative of amount of substrate cleaved by FXa for different concentrations of heparins/HS and is plotted on the Y-axes. (B) Bio-layer interferometry assay for determining the degree of PF4 binding to HS from genetically engineered CHO cells and heparin/LMWHs to PF4. Y-axis shows the PF4 binding signal (nm) at 2 doses (125 and 500 nM). PMH=porcine mucosal heparin, BMH=bovine mucosal heparin.

FIG. 9 shows the full range of concentrations used for assaying anticoagulant activity and PF4 binding of HS from genetically engineered CHO cells demonstrated in FIG. 8. (A) FXa assay where the absorbance at 405 nm is indicative of the amount of substrate cleaved by FXa using 6 different concentrations of HS. (B) Bio-layer interferometry assay for determining the degree of binding of CHO HS to PF4.

FIG. 10 shows the comparison of anticoagulant activity and PF4 binding of cellular HS and heparin/LMWHs. IC50 values for anticoagulant activity were calculated from FXa assays in FIGS. 8 and 9, and were subsequently normalized to PMH which was set to 100%. Values for PF4-binding at 500 mM PF4 for each heparin/HS sample was normalized to PMH which was set to 100%.

FIG. 11 shows results of HPLC-based disaccharide analysis of HS isolated from lysates of CHO cells. (A) Relative disaccharide compositions of CHO cells with individual KI of NDST1-4 and KO of endogenous Ndst1 and Ndst2. (B) Relative disaccharide compositions of HS from CHO cells with KI of NDST3 or NDST4 in combination with HS3ST1 or HS3ST4. Heparinase digested HS was analyzed by C18 HPLC and compared against 20 pmol disaccharide standards. Quantification of disaccharides are presented as a relative percentage of all disaccharides identified for each sample.

FIG. 12 shows results from flow cytometry analysis of antithrombin III binding to CHO cells genetically engineered as indicated. The fluorescent signal at 488 nm was recorded and mean fluorescent intensity (MFI) for each cell line is displayed in the bar chart. Experiments were performed using triplicate samples.

### STATEMENT OF THE INVENTION

The present invention provides the use of a polypeptide having heparan sulfate glucosamine 3-O-sulfotransferase 4 (HS3ST4) activity to increase the anti-coagulant activity of heparan sulfate; the use comprising providing a heparan sulfate and treating the heparan sulfate with said polypeptide to produce a heparan sulfate having increased anticoagulant activity compared to heparan sulfate which is not treated with said polypeptide; wherein the polypeptide having HS3ST4 activity has at least 80% identity to the amino acid sequence of SEQ ID NO:1.

The invention also provides a method of using a polypeptide having HS3ST4 activity to increase the anti-coagulant activity of heparan sulfate; the method comprising providing a heparan sulfate and treating the heparan sulfate with said polypeptide to produce a heparan sulfate having increased anticoagulant activity compared to heparan sulfate which is not treated with said polypeptide; wherein the polypeptide having HS3ST4 activity comprises a sequence that has at least 80% identity to SEQ ID NO:1.

Preferably, the polypeptide having HS3ST4 activity has at least 85% identity to SEQ ID NO:1; more preferably at least 86% identity; more preferably at least 87% identity; more preferably at least 87% identity; more preferably at least 88% identity; more preferably at least 89% identity; more preferably at least 90% identity; more preferably at least 91% identity; more preferably at least 92% identity; more preferably at least 93% identity; more preferably at least 94% identity; more preferably at least 95% identity; more preferably at least 96% identity; more preferably at least 97% identity; more preferably at least 98% identity; more preferably at least 99% identity; more preferably at least 99.50% identity to SEQ ID NO:1.

Preferably, the polypeptide having HS3ST4 activity has the sequence of SEQ ID NO:1.

Preferably, the polypeptide having HS3ST4 activity comprises or consists of the catalytic domain of human HS3ST4 (SEQ ID NO:4), that has at least 85% identity to SEQ ID NO:4. more preferably at least 86% identity, more preferably at least 87% identity, more preferably at least 87% identity; more preferably at least 88% identity; more preferably at least 89% identity; more preferably at least 90% identity; more preferably at least 91% identity; more preferably at least 92% identity; more preferably at least 93% identity; more preferably at least 94% identity; more preferably at least 95% identity; more preferably at least 96% identity; more preferably at least 97% identity; more preferably at least 98% identity; more preferably at least 99% identity; more preferably at least 99.50% identity to SEQ ID NO:4.

Preferably the polypeptide having HS3ST4 activity has the sequence of SEQ ID NO:4.

Heparan sulfate produced according to the use of the invention is less heterogeneous than known heparins. Heparan sulfate obtained according to the use of the invention have 3-O-sulfate groups and bind antithrombin and therefore has potent anticoagulant activity; and further has reduced or absent binding to PF4. Such heparan sulfate therefore comprise an improved safety profile such as a reduced risk of inducing heparin-induced thrombocytopenia. Further, such heparan sulfates can replace conventional heparins thereby reducing the need for animal-derived heparin. They can be used in biomedical and pharmaceutical formulations, such as coatings and drug encapsulation.

Preferably, the heparan sulfate is also treated with a polypeptide having N-Deacetylase And N-Sulfotransferase 1 (NDST1) activity wherein the polypeptide having NDST1 activity comprises a sequence according to SEQ ID NO:2 and/or is further treated with a polypeptide having N-Deacetylase And N-Sulfotransferase 2 (NDST2) activity wherein the polypeptide having NDST2 activity comprises or consists of a sequence according to SEQ ID NO:3.

Preferably, the heparan sulfate is further treated with a polypeptide having N-Deacetylase And N-Sulfotransferase 2 (NDST2) activity wherein the polypeptide having NDST2 activity comprises or consists of a sequence according to SEQ ID NO:3.

In another embodiment of the present invention, the heparan sulfate is treated with a polypeptide having HS3ST4 activity, a polypeptide having N-Deacetylase And N-Sulfotransferase 1 (NDST) activity, and a polypeptide having N-Deacetylase And N-Sulfotransferase 2 (NDST2) activity.

It has been found that polypeptides having N-Deacetylase And N-Sulfotransferase 3 (NDST3) activity (SEQ ID NO:11) or N-Deacetylase And N-Sulfotransferase 4 (NDST4) activity (SEQ ID NO:12) are particularly advantageous as they together with HS3ST4 produce the highest antithrombin Ill binding. Thus, this combination of sulfotransferases is preferred.

Accordingly, in an embodiment of the present invention, the heparan sulfate is treated with:
at least a polypeptide having HS3ST4 activity, and
one or both of a polypeptide having N-Deacetylase And N-Sulfotransferase 3 (NDST3) activity and a polypeptide having N-Deacetylase And N-Sulfotransferase 4 (NDST4) activity.

In another embodiment of the present invention, the heparan sulfate is treated with:
(i) a polypeptide having HS3ST4 activity comprising:
   (a) an amino acid sequence represented by SEQ ID NO:1, or
   (b) an amino acid sequence with at least 80% sequence identity to SEQ ID NO:1, such as at least 90% sequence identity to SEQ ID NO:1, such as at least 95% sequence identity to SEQ ID NO:1, such as at least 99% sequence identity to SEQ ID NO:1, and
(ii) a polypeptide having NDST3 activity comprising:
   (c) an amino acid sequence represented by SEQ ID NO:11, or
   (d) an amino acid sequence with at least 80% sequence identity to SEQ ID NO:11, such as at least 90% sequence identity to SEQ ID NO:11, such as at least 95% sequence identity to SEQ ID NO:11, such as at least 99% sequence identity to SEQ ID NO:11, and/or
(iii) a polypeptide having NDST4 activity comprising:
   (c) an amino acid sequence represented by SEQ ID NO:12, or
   (d) an amino acid sequence with at least 80% sequence identity to SEQ ID NO:12, such as at least 90% sequence identity to SEQ ID NO:12, such as at least 95% sequence identity to SEQ ID NO:12, such as at least 99% sequence identity to SEQ ID NO:12.

In yet another embodiment of the present invention, the heparan sulfate is treated with a polypeptide comprising an amino acid sequence represented by SEQ ID NO:1 and a polypeptide comprising an amino acid sequence represented by SEQ ID NO:11.

In a further embodiment of the present invention, the heparan sulfate is treated with a polypeptide comprising an amino acid sequence represented by SEQ ID NO:1 and a polypeptide comprising an amino acid sequence represented by SEQ ID NO:12.

In a still further embodiment of the present invention, the heparan sulfate is treated with a polypeptide comprising an amino acid sequence represented by SEQ ID NO:1, a polypeptide comprising an amino acid sequence represented by SEQ ID NO:11, and a polypeptide comprising an amino acid sequence represented by SEQ ID NO:12.

Preferably, the heparan sulfate does not exhibit any anticoagulant activity prior to treatment with the polypeptide having HS3ST4 activity.

Preferably, the sulfated heparan produced with HS3ST4 exhibits reduced binding to PF4 compared to heparan sulfate which is not treated with a polypeptide having HS3ST4 activity and/or heparan sulfate produced by another HS3ST isoenzyme such as HS3ST1, 2, 3A, 3B, 5, or 6.

Preferably, the heparan sulfate is treated with the polypeptide according to SEQ ID NO: 1 within a mammalian cell, advantageously a Chinese Hamster Ovary (CHO) cell.

Preferably the heparan sulfate that is treated within the cell is expressed by the cell, preferably endogenously.

Preferably the polypeptide having HS3ST4 activity is expressed from a coding sequence endogenous to the cell; alternatively, it is expressed from an exogenously added coding sequence. In this case the sequence encoding the polypeptide can be introduced using standard techniques as described herein.

Preferably, the cell is deficient for Chsy1, and/or CSGalNAcT1, and/or CSGalNAcT2.

Preferably, the cell is deficient in one or more 3-0 sulfotransferase enzymes and/or 2-O-sulfotransferase enzymes and/or epimerase (GLCE).

Preferably, the cell is deficient for 6-O-sulfotransferases (HS6ST1, 2 and/or 3).

Preferably, the heparan sulfate is not subject to treatment with a 6-O-sulfotransferase, especially any of HS6ST1, 2 or 3.

The mammalian cell, such as a CHO cell, may be genetically engineered to facilitate the treatment of heparan sulfate as described herein. Genetically engineering of the mammalian cells may include gene knock in (KI) and/or gene knock out (KO) of one or more genes. Preferable, combinations of KI and KO are summarized in Table 3.

Accordingly, an aspect of the present invention relates to a genetically modified mammalian cell comprising a gene encoding a polypeptide comprising an amino acid sequence represented by SEQ ID NO:1.

An embodiment of the present invention relates to the genetically modified mammalian cell as described herein, wherein the mammalian cell is a Chinese Hamster Ovary (CHO) cell.

Another embodiment of the present invention relates to the genetically modified mammalian cell as described herein, wherein the mammalian cell further comprises one or more genes selected from the group consisting of:
a gene encoding a polypeptide comprising an amino acid sequence represented by SEQ ID NO:2,
a gene encoding a polypeptide comprising an amino acid sequence represented by SEQ ID NO:3,
a gene encoding a polypeptide comprising an amino acid sequence represented by SEQ ID NO:11,
a gene encoding a polypeptide comprising an amino acid sequence represented by SEQ ID NO:12,

and combinations thereof.

Yet another embodiment of the present invention relates to the genetically modified mammalian cell as described herein, wherein the mammalian cell further comprises a gene encoding a polypeptide comprising an amino acid sequence represented by SEQ ID NO:11 and/or a gene encoding a polypeptide comprising an amino acid sequence represented by SEQ ID NO:12.

Still another embodiment of the present invention relates to the genetically modified mammalian cell as described herein, wherein the genes have been knocked in in the genetically modified mammalian cell.

A further embodiment of the present invention relates to the genetically modified mammalian cell as described herein, wherein any gene encoding HS6ST1, HS6ST2 or HS6ST3 have been knocked out.

It is to be understood that the genetically modified mammalian cell may also comprise one or more polypeptides comprising an amino acid sequence with at least 80% sequence identity, such as at least 90% sequence identity, such as at least 95% sequence identity, such as at least 99% sequence identity to any one of SEQ ID NO:1, SEQ ID NO:2, SEQ ID NO:3, SEQ ID NO:11, or SEQ ID NO:12.

Another aspect of the present invention relates to a method for producing a heparan sulfate, said method comprising the steps of:
(i) providing a genetically modified mammalian cell as described herein,
(ii) expressing heparan sulfate from said mammalian cell, and (iii) recovering said heparan sulfate,

thereby obtaining a heparan sulfate.

It is to be understood that the genetically modified mammalian cell of the method for producing a heparan sulfate is preferably a CHO cell, and it may comprise one or more of the features described for the genetically modified mammalian cell per se.

The genetically modified mammalian cell can be cultured according to common general practice which would allow synthesis and expression of the heparan sulfate. Recovering of the heparan sulfate may include lysing of the cell culture, and purification of the heparan sulfate. Lysis of the cells may be performed with any conventional means, including, but not limited to, mechanical breakage, liquid homogenization, sonication, freeze-thawing, and chemical treatment. Purification may include chromatography, such as ion-exchange chromatography and size chromatography.

Thus, an embodiment of the present invention relates to the method as described herein, wherein step (ii) of expressing heparan sulfate is immediately followed by a step of lysing the cell culture.

Another embodiment of the present invention relates to the method as described herein, wherein step (ii) of recovering said heparan sulfate comprises purification of said heparan sulfate.

The method can be used for obtaining heparan sulfate with high anti-coagulant activity and low binding affinity for PF4, which is desirable for providing an efficient pharmaceutical composition with low risk of adverse effect such as heparin-induced thrombocytopenia (HIT).

Thus, an aspect of the present invention relates to a heparan sulfate obtainable by the method as described herein.

Another aspect of the present invention relates to a pharmaceutical composition comprising the heparan sulfate.

An embodiment of the present invention relates to the pharmaceutical composition as described herein, wherein the pharmaceutical composition comprises a pharmaceutically acceptable diluent and/or one or more pharmaceutically acceptable excipients.

The pharmaceutical composition may be used as an anti-coagulant to prevent, inhibit or treat conditions for which heparin (or heparan sulfate) is typically administered. In particular, the heparan sulfate described herein decrease the clotting ability of the blood and therefore may prevent dangerous clots from forming in the blood vessels. For preventive purposes, the heparan sulfate or the pharmaceutical composition comprising the same may be administered as a blood thinner. It may also be administered to patients which are at high risk of blood clot formation, such as patients having certain types of surgery or patients laying in bed for extended periods of time.

Accordingly, an aspect of the present invention relates to the heparan sulfate or the pharmaceutical composition as described herein for use as a medicament.

Another aspect of the present invention relates to the heparan sulfate or the pharmaceutical composition as described herein for use in the prevention, inhibition or treatment of a condition related to the blood vessels, heart, kidneys, liver or lungs.

An embodiment of the present invention relates to the heparan sulfate or the pharmaceutical composition as described herein for use in the prevention, inhibition or treatment of a condition selected from the group consisting of thrombosis, acute coronary syndrome, atrial fibrillation, pulmonary embolism, cardiopulmonary bypass surgery, hemofiltration (kidney dialysis), and blood transfusion.

It is contemplated that the suggested use of the heparan sulfate or pharmaceutical composition may be as a supplementary treatment to other standard treatments. In particular, a supplement to other treatments, such as surgery, wherein there is an increased risk of blood clotting.

Another embodiment of the present invention relates to the heparan sulfate or the pharmaceutical composition for use as described herein, wherein the heparan sulfate or the pharmaceutical composition is administered intravenously or subcutaneously.

It is to be understood that the heparan sulfate or the pharmaceutical composition may similarly be used in a method of treatment.

Thus, an aspect of the present invention relates to a method of preventing, inhibiting or treating a condition related to the blood vessels, heart, kidneys, liver or lungs, wherein said method comprises administration of the heparan sulfate or the pharmaceutical composition as described herein.

An embodiment of the present invention relates to a method of preventing, inhibiting or treating thrombosis, acute coronary syndrome, atrial fibrillation, pulmonary embolism, cardiopulmonary bypass surgery, hemofiltration (kidney dialysis), or blood transfusion, wherein said method comprises administration of the heparan sulfate or the pharmaceutical composition as described herein.

The use or methods according to the invention may also be carried out in a cell-free system.

Preferably the heparan sulfate treated according to the present invention has least 25% of the anticoagulant activity exhibited by low molecular weight heparins (weight/weight), and more preferably at least 50% of the anticoagulant activity exhibited by the low molecular weight Reviparin (weight/weight), when measured using the anti-factor Xa assay described herein.

In another aspect the invention provides heparan sulfate having anticoagulant activity and having no binding affinity for PF4, or reduced binding affinity for PF4 compared to heparan sulfate produced by one or more 3-O-sulfotransferases selected from HS3ST1, 2, 3A, 3B, 5 and/or 6.

The invention also provides a method of using a polypeptide having HS3ST4 activity to increase the anti-coagulant activity of heparan sulfate; the method comprising providing a heparan sulfate and treating the heparan sulfate with said polypeptide to produce a heparan sulfate having increased anticoagulant activity compared to heparan sulfate which is not treated with said polypeptide; wherein said polypeptide comprises a sequence with least 80% identity to SEQ ID NO:1.

An embodiment of the present invention relates to the method as described herein, wherein the polypeptide having HS3ST4 activity has at least 85% identity to SEQ ID NO:1; more preferably at least 86% identity; more preferably at least 87% identity; more preferably at least 88% identity; more preferably at least 89% identity; more preferably at least 90% identity; more preferably at least 91% identity; more preferably at least 92% identity; more preferably at least 93% identity; more preferably at least 94% identity; more preferably at least 95% identity; more preferably at least 96% identity; more preferably at least 97% identity; more preferably at least 98% identity; more preferably at least 99% identity; more preferably at least 99.50% identity to SEQ ID NO:1; and most preferably has the sequence of SEQ ID NO:1.

Another embodiment of the present invention relates to the method as described herein, wherein the polypeptide comprises or consists of the catalytic domain of human HS3ST4 according to SEQ ID NO:4 or a sequence with at least 88% identity to SEQ ID NO:4; more preferably at least 89% identity; more preferably at least 90% identity; more preferably at least 91% identity; more preferably at least 92% identity; more preferably at least 93% identity; more preferably at least 94% identity; more preferably at least 95% identity; more preferably at least 96% identity; more preferably at least 97% identity; more preferably at least 98% identity; more preferably at least 99% identity; more preferably at least 99.50% identity to SEQ ID NO:4; and most preferably has the sequence of SEQ ID NO: 4.

Yet another embodiment of the present invention relates to the method as described herein, the method also comprising treating the heparan sulfate with a polypeptide having N-Deacetylase And N-Sulfotransferase (NDST) 1 activity, wherein the polypeptide having NDST1 activity comprises a sequence according to SEQ ID NO:2 and/or is also treated with a polypeptide having N-Deacetylase And N-Sulfotransferase (NDST2) activity, wherein the polypeptide having NDST2 activity comprises a sequence according to SEQ ID NO:3.

A further embodiment of the present invention relates to the method as described herein, wherein the heparan sulfate is not subject to treatment with a 6-O-sulfotransferase selected from one or more of HS6ST1, 2, and/or 3.

The invention provides more uniform, i.e., less heterogenous, compositions of heparan sulfate, substantially free from one or more contaminating GAGs, including chondroitin sulfate, dermatan sulfate, keratan sulfate and/or hyaluronic acid.

### Definitions

The term “HS3ST4 activity” refers to the action of enzymatic transfer of a sulfate group from 3′-Phosphoadenosine-5′-phosphosulfate (PAPS) to the carbon-3 position of glucosamine residues of heparan sulfate or heparin substrates by the enzyme heparan sulfate glucosamine 3-O-sulfotransferase 4. The skilled person will be able to measure the HS3ST4 activity of a polypeptide, preferably using disaccharide analysis as described herein.

The term “glycosaminoglycan” or “GAG” as used herein refers to long unbranched polysaccharides consisting of a repeating disaccharide unit. The repeating disaccharide unit consists of an amino sugar (N-acetylglucosamine or N-sulfated glucosamine) along with a uronic sugar (glucuronic acid or iduronic acid).

The term “heparin” as used herein refers to a glycosaminoglycan made of repeating disaccharide units comprising one or more of β-D-glucuronic acid (GlcA), 2-deoxy-2-acetamido-α-D-glucopyranosyl (GlcNAc), α-L-iduronic acid (IdoA), 2-O-sulfo-α-L-iduronic acid (IdoA2S), 2-deoxy-2-sulfamido-α-D-glucopyranosyl (GlcNS), 2-deoxy-2-sulfamido-α-D-glucopyranosyl-6-O-sulfate (GlcNS6S) or 2-deoxy-2-sulfamido-α-D-glucopyranosyl-3,6-O-disulfate (GlcNS3S6S) or 2-deoxy-2-sulfamido-α-D-glucopyranosyl-3-O-sulfate (GlcNS3S). The term “heparin” is used loosely in the field and may refer to heparan sulfate having anticoagulant activity. Hence, when the term “heparan sulfate having anticoagulant activity” is used herein it is intended to embrace the term “heparin” and vice versa.

The term “heparan sulfate” refers to a glycosaminoglycan composed of the same building blocks as heparin but with lower levels of sulfation. The most common disaccharide unit within heparan sulfate is composed of a glucuronic acid (GlcA) linked to N-acetylglucosamine (GlcNAc) and this typically makes up around 50% of the total disaccharide content. In porcine intestinal heparin the disaccharide IdoA(2S)-GlcNS(6S) makes up about 75%.

The term “LMWH” as used herein refers to heparin salts having an average molecular weight of less than 8,000 Da, and for which at least 60% of all chains have a molecular weight less than 8,000 Da.

The term “genetically modified cell line” as used herein refers to a cell line with specific modifications created with the editing of the genome cell line. The modification is genetically deficient in one or more gene and/or when an exogenous gene or cDNA sequence encoding a protein has been introduced. The term genetically modified cell line as used herein refers to a cell line with specific modifications created with the editing of the genome cell line. The modification is made by introducing one or more gene into a cell's genome, which is defined as genetic knock-in.

The term “heparin-induced thrombocytopenia (HIT)” refers to the development of thrombocytopenia (a low platelet count), due to the administration of various forms of heparin, an anticoagulant. HIT predisposes to thrombosis (the abnormal formation of blood clots inside a blood vessel) because platelets release microparticles that activate thrombin, thereby leading to thrombosis. When thrombosis is identified the condition is called heparin-induced thrombocytopenia and thrombosis (HITT). HIT is caused by the formation of abnormal antibodies that activate platelets. If someone receiving heparin develops new or worsening thrombosis, or if the platelet count falls, HIT can be confirmed with specific blood tests.

The term “anticoagulant” means a chemical substance that prevents or reduces coagulation of blood, prolonging the clotting time. These anticoagulants occur naturally in blood-eating animals such as leeches and mosquitoes, and anticoagulants are used in therapy for thrombotic disorders. Anticoagulants may be used in medical equipment, such as sample tubes, blood transfusion bags, heart-lung machines, and dialysis equipment. Anticoagulants inhibit specific pathways of the coagulation cascade and common anticoagulants include warfarin and heparin.

“Anticoagulant activity” can be measured by a variety of techniques well-known to persons skilled in the art. For example, anticoagulant activity may be measured using the anti-Factor Xa assay described herein

“Platelet factor 4 (PF4)” is a small cytokine belonging to the CXC chemokine family that is also known as chemokine (C-X-C motif) ligand 4 (CXCL4). PF4 is a 70-amino acid protein that is released from the alpha-granules of activated platelets and binds with high affinity to heparin. Its major physiologic role appears to be neutralization of heparin-like molecules on the endothelial surface of blood vessels, thereby inhibiting local antithrombin activity and promoting coagulation.

The “heparin:PF4 complex” is the antigen in heparin-induced thrombocytopenia, an idiosyncratic autoimmune reaction to the administration of the anticoagulant heparin. PF4 autoantibodies have also been found in patients with thrombosis and features resembling HIT but no prior administration of heparin. Antibodies against PF4 have been implicated in cases of thrombosis and thrombocytopenia subsequent to vaccination with the Oxford-AstraZeneca or the Janssen COVID-19 vaccine, which is referred to as vaccine-induced immune thrombotic thrombocytopenia (VITT).

PF4 binding affinity may be determined using bio-layer interferometry as described herein.

General DNA and molecular biology tools. Any of various techniques used for separating and recombining segments of DNA or genes, commonly by use of a restriction enzyme to cut a DNA fragment from donor DNA and inserting it into a plasmid or viral DNA. Using these techniques, DNA coding for a protein of interest is recombined/cloned (using PCR and/or restriction enzymes and DNA ligases or ligation independent methods such as USER cloning) into a plasmid (known as an expression vector), which can subsequently be introduced into a cell by transfection using a variety of transfection methods such as calcium phosphate transfection, electroporation, microinjection and liposome transfection

“Gene” refers to a DNA region (including exons and introns) encoding a gene product, as well as all DNA regions which regulate the production of the gene product, whether or not such regulatory sequences are adjacent to coding and/or transcribed sequences or situated far away from the gene which function they regulate. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions. For homologous proteins the human and rodent gene names are used inter-changeably (e.g., HS3ST4, hs3st4).

The “coding region” of a gene refers to the part of the gene that will be transcribed and translated into protein.

The “catalytic domain” of a sulfotransferase protein refers the amino acid sequence region that is required for the enzyme activity. For type II transmembrane sulfotransferase proteins this includes the C-terminal region that is highly conserved among close isoenzymes, e.g., HS3ST1-6, and that is highly conserved in evolution, e.g. between human, rodent, and fish orthologous enzymes. The sequence is approximately 250 amino acids.

The “catalytic domain” of HS3ST4 as used herein refers to such a highly conserved C-terminal region in HS3ST4, of approximately 250 amino acids. The catalytic domain of human HS3ST4 is represented by SEQ ID NO: 4, but it is appreciated that sequences having high sequence identity to SEQ ID NO: 4, such as at least 88% identity to SEQ ID NO: 4; more preferably at least 89%; more preferably at least 93%; even more preferably 96%; even more preferably 98%; even more preferably 99%; even more preferably 99.5% identity to SEQ ID NO: 4; and most preferably has the sequence of SEQ ID NO:4.

The term “Chemoenzymatic synthesis” as used herein and described in Example 4 relates to synthesis of HS polysaccharides with in vitro enzyme catalyzed reactions by using an enzymatically active form of HS3ST4 in the presence of a co-factor 3′-Phosphoadenosine-5′-phosphosulfate (PAPS) and a suitable polysaccharide for modification. Chemoenzymatic synthesis can be used for production of synthetic heparin or heparan sulfate with potent anti-coagulant activity and low or no binding to PF4 and may for example be performed in cell-free reaction systems.

The term “chimeric protein” or “fusion protein” refer to proteins created through the joining of two or more genes that originally coded for separate proteins. Recombinant chimeric or fusion proteins are created artificially by recombinant DNA technology for use in biological research or therapeutics. Translation of this chimeric or fusion gene result in a single polypeptide with functional properties derived from each of the original proteins.

Chimeric or chimera usually designate hybrid proteins made of polypeptides having different functions or physicochemical properties.

“Targeted gene modifications”, “gene editing”, “genome editing” or “genetic engineering”. Gene editing or genome editing refer to a process by which a specific chromosomal sequence is changed. The edited chromosomal sequence may comprise an insertion of at least one nucleotide, a deletion of at least one nucleotide, and/or a substitution of at least one nucleotide. Generally, genome editing inserts, replaces or removes nucleic acids from a genome using artificially engineered nucleases such as Zinc finger nucleases (ZFNs), Transcription Activator-Like Effector Nucleases (TALENs), the CRISPR/Cas system, and engineered meganuclease re-engineered homing endonucleases. Genome editing principles are broadly used and thus known to person skilled in the art.

“Endogenous” sequence/gene/protein refers to a chromosomal sequence or gene or protein that is native to the cell or originating from within the cell or organism analyzed.

“Exogenous” sequence/gene/protein refers to a chromosomal sequence that is not native to the cell, or a chromosomal sequence whose native chromosomal location is in a different location in a chromosome or originating from outside the cell or organism analyzed.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity, i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide.

The terms “polypeptide” and “protein” are used interchangeably to refer to a polymer of amino acid residues.

These terms may also refer to glycosylated variants of the “polypeptide” or “protein”, also termed “glycoprotein”.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

### Sequence Identity

Sequence identity techniques for determining nucleic acid and amino acid sequence identity are known in the art. Typically, such techniques include determining the nucleotide sequence of the mRNA for a gene and/or determining the amino acid sequence encoded thereby and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. BLASTN and BLASTP can be used to calculate alignment. Details of these programs can be found on the GenBank website and are further discussed in Example 1.

The relatedness between two amino acid sequences or between two nucleotide sequences is described by the parameter “identity”.

In one embodiment, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment, and 3) dividing the number of exact matches with the length of the reference sequence.

In one embodiment, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment, and 3) dividing the number of exact matches with the length of the longest of the two sequences.

In another embodiment, the degree of sequence identity between the query sequence and the reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the “alignment length”, where the alignment length is the length of the entire alignment including gaps and overhanging parts of the sequences.

Sequence identity comparisons can be conducted by eye, or more usually, with the aid of readily available sequence comparison programs. These commercially available computer programs use complex comparison algorithms to align two or more sequences that best reflect the evolutionary events that might have led to the difference(s) between the two or more sequences. Therefore, these algorithms operate with a scoring system rewarding alignment of identical or similar amino acids and penalising the insertion of gaps, gap extensions and alignment of non-similar amino acids. The scoring system of the comparison algorithms include:
1. i) assignment of a penalty score each time a gap is inserted (gap penalty score),
2. ii) assignment of a penalty score each time an existing gap is extended with an extra position (extension penalty score),
3. iii) assignment of high scores upon alignment of identical amino acids, and
4. iv) assignment of variable scores upon alignment of non-identical amino acids.

Most alignment programs allow the gap penalties to be modified. However, it is preferred to use the default values when using such software for sequence comparisons.

The scores given for alignment of non-identical amino acids are assigned according to a scoring matrix also called a substitution matrix. The scores provided in such substitution matrices are reflecting the fact that the likelihood of one amino acid being substituted with another during evolution varies and depends on the physical/chemical nature of the amino acid to be substituted. For example, the likelihood of a polar amino acid being substituted with another polar amino acid is higher compared to being substituted with a hydrophobic amino acid. Therefore, the scoring matrix will assign the highest score for identical amino acids, lower score for non-identical but similar amino acids and even lower score for non-identical non-similar amino acids. The most frequently used scoring matrices are the PAM matrices (Dayhoff et al. (1978), Jones et al. (1992)), the BLOSUM matrices (Henikoff and Henikoff (1992)) and the Gonnet matrix (Gonnet et al. (1992)).

Once the software has produced an alignment, it is possible to calculate % similarity and % sequence identity. The software typically does this as part of the sequence comparison and generates a numerical result.

In one embodiment, the percentage of identity of one amino acid sequence with, or to, another amino acid sequence is determined by the use of Blast with BLOSUM 62 as the substitution matrix; Gap costs: Existence: 11 Extension: 1; Compositional adjustments: Conditional compositional score matrix adjustment.

The term “chondroitin sulfate” refers to a linear polysaccharide with repeating disaccharide units that comprise one or more of N-acetylgalactosamine (GalNAc), N-acetylgalactosamine-4-sulfate (GalNAc4S), N-acetylgalactosamine-6-sulfate (GalNAc6S), N-acetylgalactosamine-4,6-disulfate (GalNAc4S6S) and β-D-glucuronic acid (GlcA), D-glucuronic acid-2-sulfate (GlcA2S), D-glucuronic acid-3-sulfate (GlcA3S), L-iduronic acid (IdoA), or L-iduronic acid-2-sulfate (IdoA2S).

The terms “sulfation pattern”, “defined pattern of sulfation”, and “defined modification pattern” as used herein refer to enzymatic modifications made to the glycosaminoglycan including but not limited to include sulfation, deacetylation, and epimerization. This also includes glycosaminoglycan compositions having a defined disaccharide composition.

The term “genetically modified cell line” as used herein refers to a cell line with specific modifications made to the genome of the cell line. In some embodiments, the cell line is mammalian. In some embodiments, the cell line is human or rodent. In some embodiments, the modifications comprise genetic knockouts, whereby the cell line becomes genetically deficient for one or more genes. In some embodiments, the modifications comprise making transgenic cell lines, whereby the cell line obtains genetic material not present in the wildtype cell line or genetic material under the control of active promoter.

All patent and non-patent references cited in the present application, are hereby incorporated by reference in their entirety.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the use of the specific polypeptides for treatment of heparan sulfate and all its features, as said polypeptides may readily be part of the methods for producing heparan sulfate, or be encoded in the genetically modified cells described herein.

The invention will now be described in further details in the following non-limiting examples.

### EXPERIMENTAL PROCEDURES

### Methods

### Heparinase Digestion

For enzymatic digestion of heparins, cellular HS and synthetic tetrasaccharides with heparinases I, II and III (IBEX Pharmaceuticals), a digestion buffer with a final concentration of 50 mM sodium acetate, 5 mM calcium acetate, pH 6.5 was used. Freshly resuspended lyophilized heparinase I was added first, heparinase III was added after 2 h, and heparinase II was added 2 h later, followed by incubation overnight at 37° C.

### C18 HPLC Disaccharide Analysis of AMAC-Labelled Disaccharides

HS from CHO cells and pharmaceutical heparins were digested with heparinases I, II and III and disaccharide products were lyophilized. The disaccharides were then labelled with AMAC by resuspension in 10 μL of 0.1 M AMAC in 3:17 (vol/vol) acetic acid/DMSO followed by incubation at room temperature for 15 min, before addition of 10 μL of 1 M NaCNBH₃ and incubation at 45° C. for 3 h. The reactions were lyophilized and excess AMAC removed by two rounds of resuspension in 500 μL acetone and pelleting by centrifugation at 20,000×g for 20 min at 4° C. Samples were dissolved in 2% acetonitrile and analyzed on a Waters® Acquity® UPLC system equipped with a fluorescence detector with a BEH C18 column (2.1×150 mm, 1.7 μm, Waters) detecting the fluorescence signal at 525 nm. A standard mix of AMAC-labeled disaccharides (20 pmol of each) was analyzed immediately prior to samples. Commercially available disaccharide standards were purchased from Iduron and Sigma-Aldrich.

### Anti-Factor Xa Assay

Human plasma ATIII (1 μM) (Sigma-Aldrich) in 50 mM Tris-HCl, 175 mM NaCl, 7.5 mM EDTA (pH 8.4) and bovine FXa (1 μM) (Sigma-Aldrich) were both diluted 1:30 in 0.9% NaCl and 8 mM FXa substrate (Sigma-Aldrich) was diluted 1:10 in 0.9% NaCl immediately prior to assay. 37.5 μL ATIII was added to each well of a 96-well plate before adding heparin/HS samples at a range of concentrations diluted to 12.5 μL in 0.9% NaCl. Mixtures were incubated for 2 min at 37° C. before addition of 37.5 μL bovine FXa followed by 1 min incubation at 37° C. 37.5 μL of FXa substrate was then added followed by incubation at 37° C. for 10 min before 37.5 μL of acetic acid was used to stop the enzymatic reaction. Absorbance was read at 405 nm using a Synergy LX plate reader (BioTek) and IC50 values used for quantification of anticoagulant activity relative to PMH were determined with the online AAT Bioquest IC50 calculator.

### Bio-Layer Interferometry

GAGs were biotinylated at their reducing end as described previously²⁴. In brief, bio-layer interferometry was carried out using streptavidin (SA) biosensors (ForteBio) hydrated for 10 min prior to use in the assay buffer (10 mM HEPES, 150 mM NaCl, 3 mM EDTA, 0.05% Tween-20, pH 7.4). Hydrated sensors were then submerged into wells of a black-walled 96-well plate containing 200 μL of biotinylated GAGs suspended at 2.5 μg/mL in assay buffer until saturated. Saturation was confirmed with an additional GAG immobilization step, where no further GAG was immobilized. Sensors were then cleaned by submersion in wells containing 200 μL of regeneration buffer (0.1 M Glycine, 1 M NaCl, 0.1% Tween, pH 9.5) and equilibrated in assay buffer. GAG coated sensors were submerged in wells containing 200 μL of PF4 re-suspended in assay buffer for 180 s (association) then transferred to wells containing assay buffer alone (dissociation), and data recorded throughout. Background binding to non-GAG coated sensors and signal produced by buffer alone was recorded and subtracted from the GAG-coated sensor signal. The maximum signal recorded during each cycle was then used as a measure of the degree of binding of PF4 to each GAG at different concentrations. Between cycles, bound PF4 was removed from GAG coated sensors using regeneration buffer and sensors were then equilibrated in assay buffer. Data was acquired using an Octet Red96 system (ForteBio) at 5 Hz and analyzed using the Octet analysis programme.

### Genetic Engineering of CHO Cells

CHOZN GS−/− (Sigma-Aldrich) cells were maintained as suspension culture in T-flasks at 37° C. and 5% CO₂ using a 1:1 mix of EX-CELL® CD CHO Fusion (Sigma-Aldrich) and BalanCD CHO Growth A (Irvine scientific), supplemented with 2 mM L-glutamine. For targeted KI at the CHO SafeHarbor locus a modified Zinc finger nuclease ObLiGare method was used as previously described^{22, 25}. Full cDNAs of coding regions of the human HS3STs (Horizon Discovery and Harvard PlasmID Database) were used, and a C-terminal S-tag or V5-tag was linked to HS3STs by PCR and constructs were further cloned into the EPB69 donor plasmid. EPB69 contained inverted CHO SafeHarbor locus ZFN binding sites flanking the CMV promoter-ORF-BGH polyA terminator, and two tDNA insulator elements flanking the ZFN-binding sites, as previously described^{22, 26}. Transfection DNA mixes contained 4.5 μg of donor plasmid DNA and 1.5 μg of each of two ZFNs tagged with GFP and Crimson, respectively. For each transfection, 1.5×10⁶ cells were electroporated using the Amaxa kit V and Amaxa Nucleofector 2B (Lonza) according to the manufacturer's instructions. 48 h after transfection, the 10-15% of cells with the highest labeling for both GFP and Crimson was enriched by FACS on a SH800 (SONY). One week later the FACS-sorted cell pool was further single-cell sorted into round-bottom 96-well plates with DMEM-F12 media (Thermo Fisher) to obtain single clones. KI clones were screened by immunocytochemistry and mono-allelic-targeted KI clones were validated by PCR with primers specific for the junction area between the donor plasmid and the Safe-Harbor locus. A primer set flanking the targeted KI locus was also used to characterize the allelic insertion status. A minimum of 3 clones were obtained for each HS3ST KI.

### Immunocytochemistry

CHO cells were washed in PBS, spotted onto Teflon printed diagnostic slides (Immuno-Cell International), air-dried, and permeabilized with ice cold acetone for 5 min. Polyclonal antibodies to S-tag (Genscript) were used 1:200 in PBS with 0.1% BSA at 4° C. overnight followed by FITC-conjugated rabbit anti-mouse IgG antibody (DAKO) 1:300 in 1×PBS with 0.1% BSA for 1 h at room temperature. A FITC-conjugated monoclonal antibody to V5-tag (Thermo Fisher) was used 1:500 in PBS with 0.1% BSA at 4° C. overnight. Slides were analyzed in Axioskop 2 plus (Zeiss) microscope and images obtained using an AxioCam MRc (Zeiss) camera.

### SDS-PAGE Western Blot

1×10⁶ cells were seeded in a T25 flask in 6 mL media and grown for 72 h, before washing the cells three times in PBS and adding 700 μL of cold RIPA buffer (50 mM Tris-HCl pH 7.5, 150 mM NaCl, 1% NP-40, 0.1% Na deoxycholate, 1 mM EDTA) containing protease inhibitor cocktail (Sigma-Aldrich). Samples were thoroughly vortexed before incubation for 20 min on ice with vortexing every 5 min followed by ultrasonication (40% amplitude) for 3×5 s with 5 s pauses using a Fisherbrand Model 120 Sonic Dismembrator (Thermo Fisher). Samples were centrifuged at 20,000×g at 4° C. for 15 min, and the protein concentration of the supernatant was measured using a BCA protein assay kit (Thermo Scientific). 10 μg protein or the corresponding fraction of media used for culturing cells to obtain 10 μg protein was mixed with 10 mM DDT and 1× loading buffer, heated to 90° C. for 10 min, and separated on NuPage 4-12% Bis-Tris gels (Thermo Fisher). Proteins were transferred to nitrocellulose membranes at 320 mA for 60 min in MES buffer with 20% methanol. Membranes were blocked with 5% skimmed milk in TBS-T for 60 min before incubated with either HRP-conjugated antibodies to V5-tag (Thermo Fisher) or S-tag in TBS-T with 5% skimmed milk at 4° C. overnight. Membranes were washed 3×5 min in TBS-T followed by incubation of S-tag membranes with HRP-conjugated rabbit anti-mouse IgG antibody (DAKO) in 5% skimmed milk in TBS-T for 1 h at room temperature and washing 3×5 min in TBS-T. Pierce™ ECL Plus Western Blotting Substrate (Thermo Fisher) was used according to the manufacturer's instructions and images were captured using the ImageQuant™ Las 4000 (GE Healthcare).

Extraction and Purification of GAGs from CHO Cells

Cells were washed in PBS and diluted to 1×10⁷ cells/mL in 50 mM Tris-HCl pH 7.4, 10 mM CaCl₂, 0.1% Triton X-100. Pronase (Roche) was added (1 mg/mL) and reactions were incubated overnight rotating tray in an incubator set at 37° C. followed by heat inactivation. 5 mM MgCl₂ and 1 μg/mL DNasel (Sigma-Aldrich) was added and samples were incubated at 37° C. for 4 h. Samples were treated with 10 μg/mL RNaseA (Sigma-Aldrich) and 5 mM EDTA at 37° C. for 2 h, followed by 0.5 mU/mL neuraminidase (Sigma-Aldrich) at 37° C. overnight. For cells expressing CS, 20 mU/mL chABC was added and samples were incubated at 37° C. for 4 h. Samples were again incubated with pronase at 1 mg/mL for overnight digestion at 37° C. Samples were acidified to pH 4-5 with acetic acid, centrifuged at 20,000×g for 20 min, filtered through 0.45 μm filters, and isolated on HiTrap DEAE FF columns (5 mL, GE Healthcare). Columns were equilibrated with 20 mM NaOAc and 0.5 M NaCl (pH 5.0) and samples were eluted with 1.25 M NaCl. GAGs were precipitated by addition of cold NaOAc-saturated 100% ethanol (3:1 vol/vol), centrifuged at 20,000×g for 20 min at 4° C., and the pellets were dried on a speed-vac.

Samples were re-suspended in deionized water and further purified using a Discovery BIO Wide Pore C5-5 (Sigma-Aldrich) and desalted on 1 mL HiTrap desalting columns (GE Healthcare).

### EXAMPLES

The purpose of the following examples is given as an illustration of various aspects of the invention and are thus not meant to limit the present invention in any way. Along with the present examples the methods described herein are presently representative of preferred aspects, are exemplary, and are not intended as limitations on the scope of the invention. Changes therein and other uses which are encompassed within the spirit of the invention as defined by the scope of the claims will occur to those skilled in the art.

### Example 1: Genetic Engineering of HS3STs into CHO and Mammalian Cells

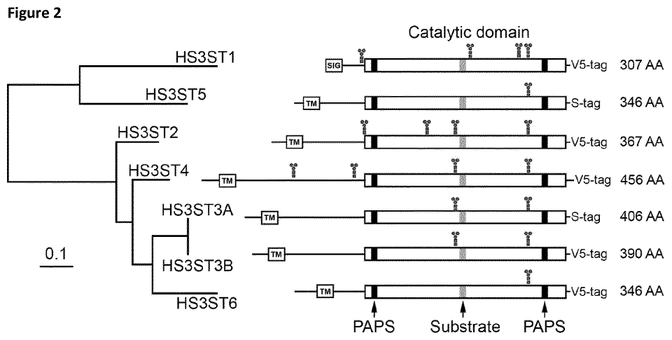

The addition of 3-O-sulfate groups to HS is catalyzed by seven distinct isoenzymes grouping by sequence similarity into a subfamily of closely homologous HS3ST1 and 5, more distinct isoenzymes HS3ST2 and HS3ST4, and a subfamily of HS3ST3A, HS3ST3B, and HS3ST6 (FIG. 2). Comparing the amino acid sequence of the full coding region of HS3ST4 between representative species covering a wide range of evolution from human to fish shows that HS3ST4 is highly conserved in evolution with 65.74% sequence identity between human and zebrafish (Table 1).

TABLE 1
Amino acid sequence similarity of the full coding
sequences of HS3ST4 from selected species.

| Accession number | Species | % Amino acid identity | % Query cover |
|---|---|---|---|
| NP_006031.2 | Homo sapiens | 100 (456/456) | 100 |
| | (human) | | |
| XP_016785153.1 | Pan troglodytes | 99.12 (452/456) | 100 |
| | (chimpanzee) | | |
| e | Desmodus rotundus | 95.64 (307/321) | 70 |
| | (common vampire bat) | | |
| XP_038524728.1 | Canis lupus familiaris | 89.98 (395/439) | 94 |
| | (dog) | | |
| XP_017445955.2 | Rattus norvegicus | 88.68 (384/433) | 94 |
| | (brown rat) | | |
| NP_001239001.1 | Mus musculus | 88.22 (382/433) | 94 |
| | (house mouse) | | |
| XP_044836108.1 | Mauremys mutica | 75.45 (338/448) | 96 |
| | (yellow pond turtle) | | |
| XP_015149991.2 | Gallus gallus | 72.17 (319/442) | 96 |
| | (red junglefowl) | | |
| XP_044186045.1 | Thunnus albacares | 66.82 (284/425) | 92 |
| | (yellowfin tuna) | | |
| NP_001074058.1 | Danio rerio | 65.74 (284/432) | 92 |
| | (zebrafish) | | |

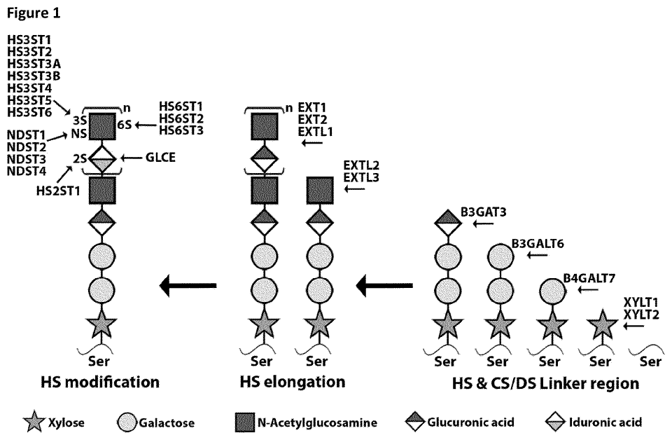

The analysis of amino acid sequences of full coding regions of the seven human HS3STs and the evolutionary analysis of select amino acid sequences of full coding regions of HS3ST4 show that the sequence similarity is highest in the central and C-terminal regions of these coding regions, and this outlines the predicted common catalytic domains of the sulfotransferase enzymes. Comparing the amino acid sequence of the predicted catalytic domain of HS3ST4 between species shows that HS3ST4 is highly conserved in evolution with 89.58% sequence identity between human and zebrafish (FIG. 1, Table 2). For table 1 and table 2 alignments were made using BLASTP as available at https://last.ncbi.nlm.nih.gov/Blast.cgi?PROGRAM=blastp&PAGE_TYPE=BlastSearch&LINK_LOC=blasthome on 15 Dec. 2021, with the following parameters: Matrix: BLOSUM62, Gap costs: Existence: 11 Extension: 1; Compositional adjustments: Conditional compositional score matrix adjustment.

TABLE 2
Amino acid sequence similarity of the catalytic
domains of HS3ST4 from selected species.

| Accession number | Species | % Amino acid identity | % Query cover |
|---|---|---|---|
| NP_006031.2 | Homo sapiens | 100 (262/262) | 100 |
| | (human) | | |
| NP_001239001.1 | Mus musculus | 99.62 (261/262) | 100 |
| | (house mouse) | | |
| XP_017445955.2 | Rattus norvegicus | 99.62 (261/262) | 100 |
| | (brown rat) | | |
| XP_016785153.1 | Pan troglodytes | 99.62 (261/262) | 100 |
| | (chimpanzee) | | |
| XP_024416119.1 | Desmodus rotundus | 98.47 (258/262) | 100 |
| | (common vampire bat) | | |
| XP_038524728.1 | Canis lupus familiaris | 97.33 (255/262) | 100 |
| | (dog) | | |
| XP_044836108.1 | Mauremys mutica | 96.95 (254/262) | 100 |
| | (yellow pond turtle) | | |
| XP_015149991.2 | Gallus gallus | 93.89 (246/262) | 100 |
| | (red junglefowl) | | |
| XP_044186045.1 | Thunnus albacares | 89.96 (233/262) | 98 |
| | (yellowfin tuna) | | |
| NP_001074058.1 | Danio rerio | 89.58 (232/262) | 98 |
| | (zebrafish) | | |

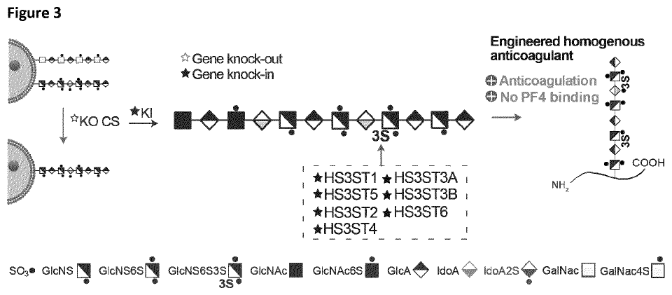

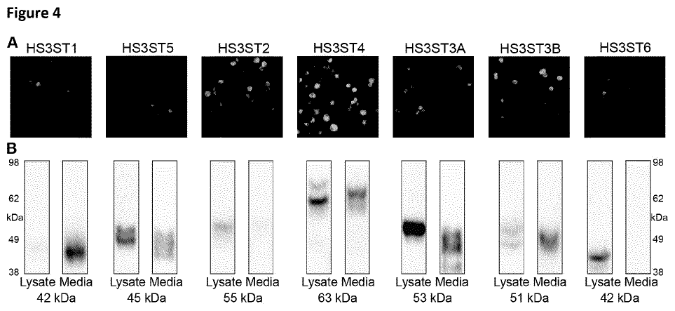

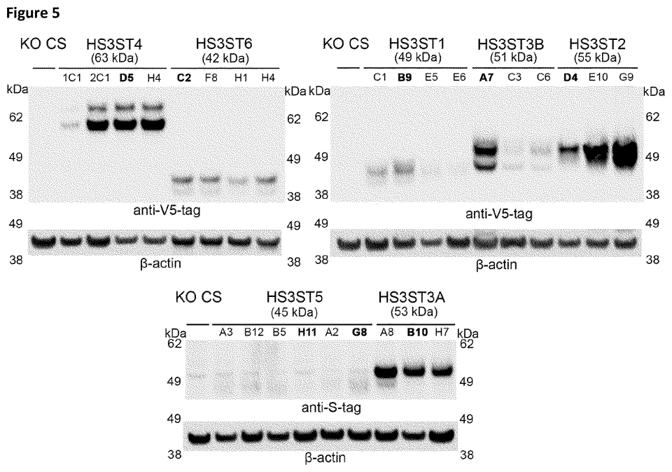

To systematically analyze the importance of 3-O-sulfation for the structure and bioactivity of heparin/HS, we employed a genetic engineering approach to individually KI all seven human HS3STs (cDNA plasmids including the full coding regions) into CHO cells (FIG. 3). CHO cells were selected as they are devoid of background HS3ST expression and significant biosynthesis of 3-O-sulfated HS. Furthermore, CHO cells express both HS and CS, and to avoid the presence of CS as a contaminating GAG, we used a genetically engineered cell line with knock-out (KO) of CSGalNAcT1/CSGalNAcT2/Chsy1 (designated as CHO KO CS), where KI of the seven human 3-O-sulfotransferases was performed by site-directed ZFN gene KI. Expression of the HS3ST enzymes was confirmed by immunocytochemistry and SDS-PAGE Western blot analysis (FIGS. 4 and 5). HS3ST1 lacks an apparent transmembrane domain and was predominantly detected as a secreted protein in the culture media. HS3ST2/6 were observed only in the cell lysate and HS3ST5/4/3A/3B were observed in both lysate and culture media, potentially indicating extracellular activities of these enzymes. The presence of sulfotransferases in the media may be due to proteolytic cleavage of the transmembrane domains and secretion of the soluble catalytical active domains comprised of the C-terminal part (FIG. 2), which has previously been shown to result in secretion of HS3STs, NDSTs and HS6STs²⁷.

### Example 2: 3-O-Sulfotransferase-Dependent Alterations in HS Profiles of Engineered Cells

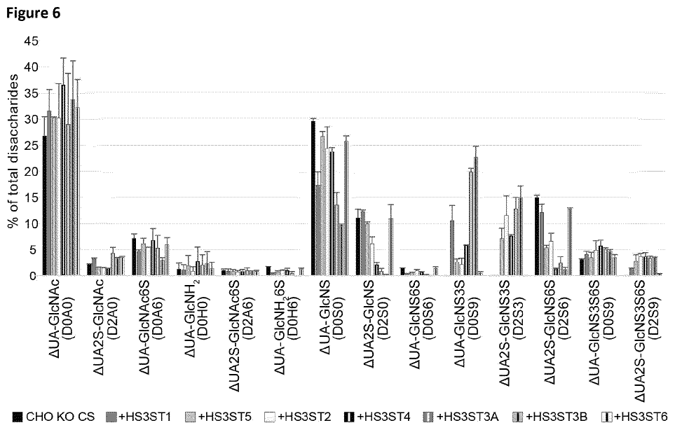

Disaccharide analysis was performed on HS from our genetically engineered HS3ST-expressing CHO cells to study the properties of the seven HS3STs by analyzing the HS at the disaccharide level (FIG. 6). HPLC disaccharide analysis revealed distinct differences for most KI clones compared to the parent CHO cell. HS3ST1 primarily introduced ΔUA-GlcNS3S (DOS3), suggesting ΔUA-GlcNS (DOSO) is the preferred substrate. In contrast, the related HS3ST5 mainly introduced ΔUA2S-GlcNS3S (D2S3) and ΔUA2S-GlcNS3S6S (D2S9). HS3ST2 displayed a preference for 2-O-sulfated epitopes as ΔUA2S-GlcNS3S (D2S3) was the main 3-O-sulfated disaccharide; however, it is interesting to note that HS3ST2 generated minimal ΔUA-GlcNS3S (DOS3) units. HS3ST4 displayed high levels of all four 3-O- and N-sulfated disaccharides, indicating that the enzyme is active on a wider range of substrates. As expected, HS3ST3A/3B showed similar disaccharide profiles, and in this case ΔUA2S-GlcNS3S (D2S3) and ΔUA-GlcNS3S (DOS3) were the predominant 3-O-sulfated disaccharides. CHO cell HS from HS3ST6 showed a disaccharide profile very similar to the parental clone with minute levels of 3-O-sulfation detected. Taken together, the analysis of the HS3STs indicate that they all have preferences for N-sulfated substrates, and that HS3ST5/2 has a strong preference for 2-O-sulfated substrates, while HS3ST3A/3B/4 has more promiscuous substrate specificity for N/2-O/6-O-sulfated substrates.

### Example 3: Divergent Bioactivities of HS from HS3ST-Expressing Cells

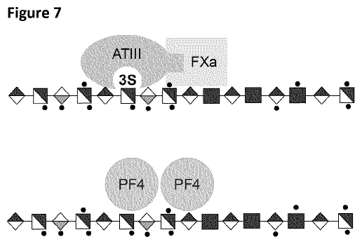

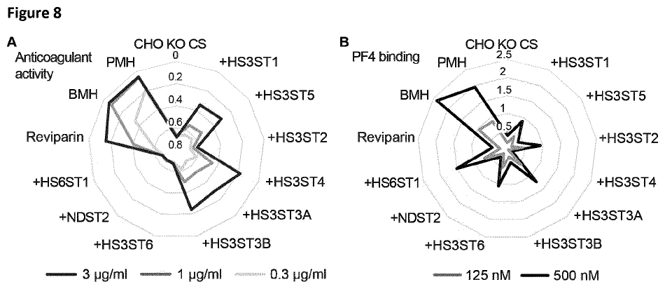

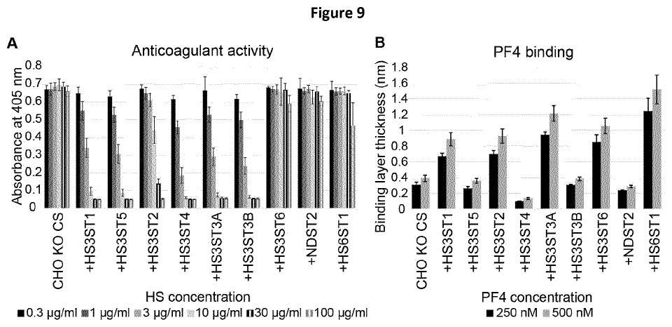

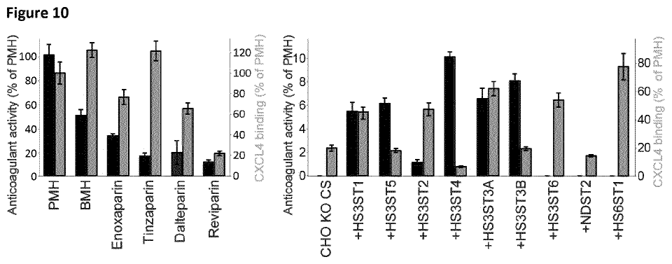

In order to relate different 3-O-sulfated HS structures derived by the action of the 3-O-sulfotransferase family to resulting bioactivities, and to also exploit this approach for initial development of a cell-based heparin/HS with low PF4 binding, we tested our HS3ST library of CHO cells for anticoagulant activity and PF4 binding (FIG. 7). We first analyzed the anticoagulant activity of HS isolated from the engineered CHO cells and compared these to clinical heparin/LMWHs using anti-FXa assays (FIGS. 8A and 9A). HS from CHO KO CS demonstrated no measurable anticoagulant activity, whilst CHO cell HS produced by HS3ST1 showed anticoagulant activity in agreement with previous studies²⁸, Interestingly, CHO cell HS from HS3ST5/4/3A/3B demonstrated even higher levels of activity than HS3ST1, whilst CHO cell HS from HS3ST2 had comparatively low levels and HS3ST6 demonstrated no anticoagulant activity. For enabling comparison of anticoagulant activity between the cellular derived HS and heparins we calculated IC50 values from the FXa data, where lower IC50 values indicates more potent activity (FIG. 10). Remarkably, the FXa activities of some of the CHO cell derived HS were comparable to the activities found for some of the LMWHs. Thus, HS produced by CHO cells with KI of HS3ST4 exhibit almost 80% of the activity found for the LMWH Reviparin. In clinical practice the low IC50 of heparin is not necessary to achieve the anticoagulant effect, as LMWHs are efficient in the treatment of thrombosis and are clinically more commonly used than heparin. HS from CHO cells with KI of N-sulfation (CHO KI NDST2) and 6-O-sulfation (CHO KI HS6ST1)²³ did not demonstrate anticoagulant activity, emphasizing the importance of the 3-O-sulfation over N-/6-O-sulfation.

HIT is a potential life threatening, immune-mediated adverse drug reaction to heparin, due to formation of PF4-heparin complexes. We tested PF4 binding of HS from the HS3ST1-6 KI cell lines (FIGS. 8B, 9B, and 10), and found that HS from CHO cells expressing HS3ST4 demonstrated particularly low PF4 binding, despite exhibiting the highest anticoagulant activity. Thus, surprisingly this isoenzyme HS3ST4 is a potential candidate to use for bioengineering heparin with potent anticoagulant activity and reduced potential to cause HIT. CHO cell HS from HS3ST5/3B also demonstrated comparatively low binding to PF4, while CHO cell HS from HS3ST1/2/3A/6 exhibited increased binding compared to CHO KO CS. HS from CHO cells with KI of HS6ST1 showed the strongest PF4 binding of all cellular HS, indicating that not only N-/2-O-sulfation, but also 6-O-sulfation could be important for PF4-heparin complex formation. This data demonstrates the ability of the cell-based strategy to both identify and optimize HS bioactivities associated with distinct biosynthetic enzyme combinations.

Table 3 summarizes desirable combinatorial genetic engineering designs applicable to CHO cells including knock in (KI) of an animal HS3ST4. For other mammalian cells endogenously expressing HS3ST1, 2, 3A, 3B, 5, and/or 6 it is desirable to knock out (KO) one or more of these when implementing the desirable engineering designs in these. The HS3ST4 enzyme is highly conserved in evolution and the orthologous enzyme clearly identifiable by sequence analysis in all animals down to fish (Tables 1 and 2). KI of HS3ST4 derived from any of these species can thus be used to engineer cells. The catalytic domain of HS3ST4 is clearly identifiable by sequence analysis and for example comprises amino acids 130-453 of the human HS3ST4.

TABLE 3
Overview of desirable gene engineering designs applicable to CHO cells for production
of heparan sulfate with anticoagulant activity and with low or no PF4 binding.

| | Cell Line ID | Gene KO | Gene KI | Enzymes |
|---|---|---|---|---|
| 1 | CHO | | HS3ST4 | HS 3-O-sulfation Added |
| 2 | CHO | | HS3ST4, NDST1 | HS 3-O-sulfation Added |
| | | | | HS N-sulfation Added |
| 3 | CHO | | HS3ST4, NDST2 | HS 3-O-sulfation Added |
| | | | | HS N-sulfation Added |
| 4 | CHO | | HS3ST4, NDST3 | HS 3-O-sulfation Added |
| | | | | HS N-sulfation Added |
| 5 | CHO | | HS3ST4, NDST4 | HS 3-O-sulfation Added |
| | | | | HS N-sulfation Added |
| 6 | CHO | Hs6st1-3 | HS3ST4, NDST1 | HS 6-O-sulfation deficient |
| | | | | HS 3-O-sulfation KI |
| | | | | HS N-sulfation KI |
| 7 | CHO | Hs6st1-3 | HS3ST4, NDST2 | HS 6-O-sulfation deficient |
| | | | | HS 3-O-sulfation KI |
| | | | | HS N-sulfation KI |
| 8 | CHO | Hs6st1-3 | HS3ST4, NDST3 | HS 6-O-sulfation deficient |
| | | | | HS 3-O-sulfation KI |
| | | | | HS N-sulfation KI |

### Example 4: Chemoenzymatic Synthesis of 3-O-Sulfated Heparin/HS Using HS3ST4

Chemoenzymatic methods for synthesis of heparin sulfate and heparin are well described in the literature, see for example Liu and Lindhardt²⁹ and Zhang et al³⁰. Chemoenzymatic synthesis of HS polysaccharides employing an enzymatically active form of HS3ST4 using appropriate saccharides and 3′-Phosphoadenosine-5′-phosphosulfate (PAPS) donor substrates is preferable for production of synthetic heparin/HS with potent anti-coagulant activity and low or no binding to PF4. Active forms of HS3ST4 are comprised of the full coding sequence of the HS3ST4 gene from any species and N-terminal truncated versions of these that includes at least the predicted catalytic domains identified as outlined in FIG. 2 and Table 2. Chimeric HS3ST4 fusion proteins containing the catalytic domain of an HS3ST4 sequence and another protein sequence or protein domain may be produced recombinantly in cells and used as enzyme source. Recombinant active forms of HS3ST4 are produced in eukaryotic or prokaryotic cells and the cell preparation or purified active enzyme protein used in enzyme reactions with HS polysaccharide acceptor substrates and PAPs donor substrates. Preferable HS polysaccharide substrates include a 12-mer oligosaccharide containing the GlcNS-IdoA2S-disaccharide repeating unit. The enzyme reaction may for example include 20 mg of HS substrate incubated with 0.15 mmol of PAPS and purified HS3ST4 in a volume of 200 mL of the reaction buffer containing 50 mM Tris (pH 7.2), 2 mM MnCl2, and 2 mM CaCl2. After incubation at 37° C. for 24 hrs the product can be purified by a 30 mL Giga Q column (Tosohaas Bioscience) and eluted by 0 to 1 M NaCl in 20 mM NaOAc (pH 5.0).

### Example 5: NDST3 and/or NDST4 in Combination with HS3ST4 Induces High Anticoagulant Activity

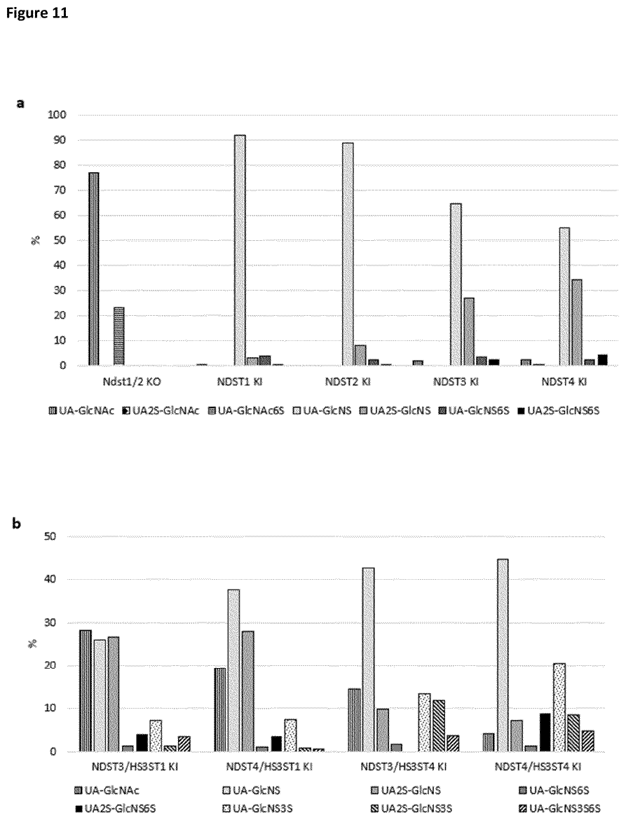

To systematically analyze the importance of N- and 2-O-sulfation for the structure and bioactivity of heparin/HS, we employed further engineering to individually KI all four human NDST1-4 (cDNA including the full coding regions) into CHO cells with and without stable KI of HS3ST1 or HS3ST4 (FIG. 11, Table 3). The endogenously expressed Ndst1 and Ndst2 genes were inactivated by KO prior to the KI experiments. Disaccharide analysis of HS isolated from CHO cells with stable KI of the individual NDSTs revealed marked induction of synthesis of N-sulfated (UA-GlcNS) and to varying degree N- and 2-O-sulfated (UA2S-GlcNS) disaccharides (FIG. 11A). Remarkably, cells with KI of NDST3 and NDST4 produced substantially higher relative levels of the N- and 2-O-sulfated (UA2S-GlcNS) disaccharide, while NDST1 and NDST2 largely only produced the N-sulfated (UA-GlcNS) disaccharide. Next, we determined the effects of combinatorial KI of NDST1-4 and HS3ST4, and as shown in FIG. 11B KI of NDST3 or NDST4 together with either HS3ST1 or HS3ST4 resulted in distinct disaccharide profiles. KI of NDST3/4 with HS3ST1 produced HS with predominant N- and 2-O-sulfated disaccharides with a low degree of 3-O-sulfated disaccharides, whereas KI of NDST3/4 with HS3ST4 resulted in marked increase in 3-O-sulfated disaccharides (UA-GlcNS3S, UA2S-GlcNS3S) (FIG. 11B).

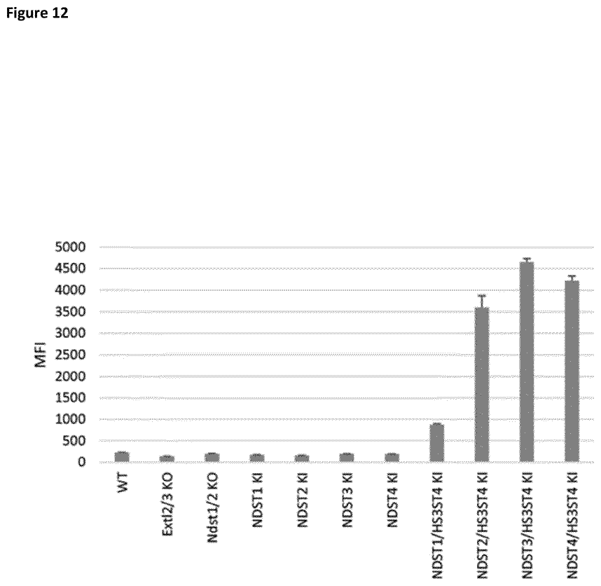

Further analysis of antithrombin III binding to the genetically engineered CHO cells by flow cytometry assays revealed that NDST3 or NDST4 in combination with HS3ST4 results in the highest antithrombin III binding (FIG. 12). Genetically engineered CHO cells were harvested and washed in 1×PBS, before incubation with 500 mM antithrombin III (Aniara) in 0.5% BSA in 1×PBS for 40 min at 4° C. Cells were washed in 0.5% BSA in 1×PBS before incubation with 10 μg/ml biotinylated human serpin-c1 antibody (R&D systems) in 0.5% BSA in 1×PBS for 30 min at 4° C. Cells were washed in 0.5% BSA in 1×PBS and further incubated with 1:2000 Alexa Fluor 488-streptavidin (Invitrogen) in 0.5% BSA in 1×PBS for 30 min at 4° C. Cells were washed and resuspended in 0.5% BSA in 1×PBS and fluorescent intensity was measured at 488 nm on a SONY spectral analyzer. These results suggest that a preferable combination of sulfotransferases for the synthesis and production of heparin includes NDST3 or NDST4 together with HS3ST4.

### REFERENCES CITED

1. Xu, D. & Esko, J. D. Demystifying heparan sulfate-protein interactions. Annu Rev Biochem 83, 129-157 (2014).
2. Cadwallader, A. B. & Yost, H. J. Combinatorial expression patterns of heparan sulfate sulfotransferases in zebrafish: I. The 3-O-sulfotransferase family. Dev Dyn 235, 3423-3431 (2006).
3. Lawrence, R. et al. The principal neuronal gD-type 3-O-sulfotransferases and their products in central and peripheral nervous system tissues. Matrix Biol 26, 442-455 (2007).
4. Thacker, B. E., Xu, D., Lawrence, R. & Esko, J. D. Heparan sulfate 3-O-sulfation: a rare modification in search of a function. Matrix Biol 35, 60-72 (2014).
5. Liu, J., Shworak, N. W., Fritze, L. M., Edelberg, J. M. & Rosenberg, R. D. Purification of heparan sulfate D-glucosaminyl 3-O-sulfotransferase. J Biol Chem 271, 27072-27082 (1996).
6. Xia, G. et al. Heparan sulfate 3-O-sulfotransferase isoform 5 generates both an antithrombin-binding site and an entry receptor for herpes simplex virus, type 1. J Biol Chem 277, 37912-37919 (2002).
7. Meissen, J. K. et al. Differentiation of 3-O-sulfated heparin disaccharide isomers: identification of structural aspects of the heparin CCL2 binding motif. J Am Soc Mass Spectrom 20, 652-657 (2009).
8. Oduah, E. I., Linhardt, R. J. & Sharfstein, S. T. Heparin: Past, Present, and Future. Pharmaceuticals (Basel) 9 (2016).
9. Fu, L. et al. Structural characterization of pharmaceutical heparins prepared from different animal tissues. J Pharm Sci 102, 1447-1457 (2013).
10. Liu, H., Zhang, Z. & Linhardt, R. J. Lessons learned from the contamination of heparin. Nat Prod Rep 26, 313-321 (2009).
11. Huntington, J. A. Mechanisms of glycosaminoglycan activation of the serpins in hemostasis. J Thromb Haemost 1, 1535-1549 (2003).
12. Kim, Y. S. & Linhardt, R. J. Structural features of heparin and their effect on heparin cofactor II mediated inhibition of thrombin. Thromb Res 53, 55-71 (1989).
13. Petitou, M. et al. The synthetic pentasaccharide fondaparinux: first in the class of antithrombotic agents that selectively inhibit coagulation factor Xa. Semin Thromb Hemost 28, 393-402 (2002).
14. Atha, D. H., Lormeau, J. C., Petitou, M., Rosenberg, R. D. & Choay, J. Contribution of monosaccharide residues in heparin binding to antithrombin III. Biochemistry 24, 6723-6729 (1985).
15. HajMohammadi, S. et al. Normal levels of anticoagulant heparan sulfate are not essential for normal hemostasis. J Clin Invest 111, 989-999 (2003).
16. Zhang, L. et al. The retinoic acid and cAMP-dependent up-regulation of 3-O-sulfotransferase-1 leads to a dramatic augmentation of anticoagulantly active heparan sulfate biosynthesis in F9 embryonal carcinoma cells. J Biol Chem 273, 27998-28003 (1998).
17. Rauova, L. et al. Ultralarge complexes of PF4 and heparin are central to the pathogenesis of heparin-induced thrombocytopenia. Blood 105, 131-138 (2005).
18. Dyer, D. P., Salanga, C. L., Volkman, B. F., Kawamura, T. & Handel, T. M. The dependence of chemokine-glycosaminoglycan interactions on chemokine oligomerization. Glycobiology 26, 312-326 (2016).
19. Ahmed, I., Majeed, A. & Powell, R. Heparin induced thrombocytopenia: diagnosis and management update. Postgrad Med J 83, 575-582 (2007).
20. Stringer, S. E. & Gallagher, J. T. Specific binding of the chemokine platelet factor 4 to heparan sulfate. J Biol Chem 272, 20508-20514 (1997).
21. Lord, M. S. et al. Bioengineered human heparin with anticoagulant activity. Metab Eng 38, 105-114 (2016).
22. Yang, Z. et al. Engineered CHO cells for production of diverse, homogeneous glycoproteins. Nat Biotechnol 33, 842-844 (2015).
23. Chen, Y. H. et al. The GAGOme: a cell-based library of displayed glycosaminoglycans. Nat Methods 15, 881-888 (2018).
24. Thakar, D. et al. A quartz crystal microbalance method to study the terminal functionalization of glycosaminoglycans. Chem Commun (Camb) 50, 15148-15151 (2014).
25. Maresca, M., Lin, V. G., Guo, N. & Yang, Y. Obligate ligation-gated recombination (ObLiGaRe): custom-designed nuclease-mediated targeted integration through nonhomologous end joining. Genome Res 23, 539-546 (2013).
26. Hintze, J. et al. Probing the contribution of individual polypeptide GalNAc-transferase isoforms to the O-glycoproteome by inducible expression in isogenic cell lines. J Biol Chem 293, 19064-19077 (2018).
27. Kuhn, P. H. et al. Secretome analysis identifies novel signal Peptide peptidase-like 3 (Sppl3) substrates and reveals a role of Sppl3 in multiple Golgi glycosylation pathways. Mol Cell Proteomics 14, 1584-1598 (2015).
28. Datta, P. et al. Bioengineered Chinese hamster ovary cells with Golgi-targeted 3-O-sulfotransferase-1 biosynthesize heparan sulfate with an antithrombin-binding site. J Biol Chem 288, 37308-37318 (2013).
29. Liu, J. & Linhardt, R. J. Chemoenzymatic synthesis of heparan sulfate and heparin. Nat Prod Rep 31, 1676-1685 (2014).
30. Zhang, X., Lin, L., Huang, H. & Linhardt, R. J. Chemoenzymatic Synthesis of Glycosaminoglycans. Acc Chem Res 53, 335-346 (2020).

1. A method of using a polypeptide having heparan sulfate glucosamine 3-O-sulfotransferase 4 (HS3ST4) activity to increase the anti-coagulant activity of heparan sulfate; the method comprising providing a heparan sulfate and treating the heparan sulfate with said polypeptide to produce a heparan sulfate having increased anticoagulant activity compared to heparan sulfate which is not treated with said polypeptide; wherein said polypeptide comprises a sequence with at least 80% identity to SEQ ID NO:1.

2-20. (canceled)

21. The method according to claim 1, wherein the polypeptide having HS3ST4 activity has at least 85% identity to SEQ ID NO:1.

22. The method according to claim 1, wherein the polypeptide having HS3ST4 activity comprises the catalytic domain of human HS3ST4 according to SEQ ID NO:4 or a sequence having at least 88% identity to SEQ ID NO: 4.

23. The method according to claim 1, wherein the heparan sulfate is also treated with a polypeptide having N-Deacetylase And N-Sulfotransferase (NDST) 1 activity wherein the polypeptide having NDST1 activity comprises a sequence according to SEQ ID NO:2.

24. The method according to claim 1, wherein the heparan sulfate is also treated with a polypeptide having N-Deacetylase And N-Sulfotransferase (NDST2) activity wherein the polypeptide having NDST2 activity comprises a sequence according to SEQ ID NO:3.

25. The method according to claim 1, wherein the heparan sulfate is treated with a polypeptide having N-Deacetylase And N-Sulfotransferase 3 (NDST3) activity wherein the polypeptide having NDST3 activity comprises a sequence according to SEQ ID NO:11.

26. The method according to claim 1, wherein the heparan sulfate is treated with a polypeptide having N-Deacetylase And N-Sulfotransferase 4 (NDST4) activity wherein the polypeptide having NDST4 activity comprises a sequence according to SEQ ID NO:12.

27. The method according to claim 1, wherein the heparan sulfate does not exhibit anticoagulant activity prior to treatment with the polypeptide having HS3ST4 activity.

28. The method according to claim 1, wherein the sulfated heparan produced with HS3ST4 exhibits reduced binding to PF4 compared to heparan sulfate which is not treated with a polypeptide having HS3ST4 activity.

29. The method according to claim 1, wherein the heparan sulfate is treated with the polypeptide having HS3ST4 activity within a mammalian cell, preferably a Chinese Hamster Ovary (CHO) cell.

30. The method according to claim 29, wherein the cell is deficient for Chsy1, and/or CSGalNAcT1, and/or CSGalNAcT2 and/or one or more of 6-O-sulfotransferases HS6ST1, 2, or 3.

31. A genetically modified mammalian cell comprising a gene encoding a polypeptide comprising:
an amino acid sequence represented by SEQ ID NO:1, or
an amino acid sequence with at least 80% sequence identity to SEQ ID NO:1, such as at least 90% sequence identity to SEQ ID NO:1, such as at least 95% sequence identity to SEQ ID NO:1, such as at least 99% sequence identity to SEQ ID NO:1.

32. The genetically modified mammalian cell according to claim 31, wherein the mammalian cell further comprises one or more genes selected from the group consisting of:
(a) a gene encoding a polypeptide comprising an amino acid sequence represented by SEQ ID NO:2,
(b) a gene encoding a polypeptide comprising an amino acid sequence represented by SEQ ID NO:3,
(c) a gene encoding a polypeptide comprising an amino acid sequence represented by SEQ ID NO:11, and
(d) a gene encoding a polypeptide comprising an amino acid sequence represented by SEQ ID NO:12,
and combinations thereof.

33. The genetically modified mammalian cell according to claim 31, wherein the mammalian cell further comprises a gene encoding a polypeptide comprising an amino acid sequence represented by SEQ ID NO:11 and/or a gene encoding a polypeptide comprising an amino acid sequence represented by SEQ ID NO:12.

34. A method for producing a heparan sulfate, said method comprising the steps of:
(i) providing a genetically modified mammalian cell according to claim 31,
(ii) expressing heparan sulfate from said mammalian cell, and
(iii) recovering said heparan sulfate,
thereby obtaining the heparan sulfate.

35. A heparan sulfate obtainable by the method according to claim 34.

36. A pharmaceutical composition comprising the heparan sulfate according to claim 35.

37. A method of preventing, inhibiting or treating a condition related to the blood vessels, heart, kidneys, or lungs, wherein said method comprises administration of the pharmaceutical composition according to claim 36.

38. A method of preventing, inhibiting or treating thrombosis, acute coronary syndrome, atrial fibrillation, pulmonary embolism, cardiopulmonary bypass surgery, hemofiltration (kidney dialysis), or blood transfusion, wherein said method comprises administration of the pharmaceutical composition according to claim 36.

39. Heparan sulfate having anticoagulant activity and having no binding affinity for PF4, or reduced binding affinity for PF4 compared to heparan sulfate produced by one or more 3-O-sulfotransferases selected from HS3ST1, 2, 3A, 3B, 5 and/or 6.


SEQUENCE LISTING (12 sequences)

SEQ ID NO: 1 - protein, 456 aa, Homo sapiens
MARWPAPPPPPPPPPPLAAPPPPGASAKGPPARKLLFMCTLSLSVTYLCYSLLGGSGSLQFPLALQESPGAAAEPPPSPPPPSLLPTPVRLGAPSQPPAPPPLDNASHGEPPEPPEQPAAPGTDGWGLPSGGGGAQDAWLRTPLAPSEMITAQSALPEREAQESSTTDEDLAGRRAANGSSERGGAVSTPDYGEKKLPQALIIGVKKGGTRALLEAIRVHPDVRAVGVEPHFFDRNYEKGLEWYRNVMPKTLDGQITMEKTPSYFVTNEAPKRIHSMAKDIKLIVVVRNPVTRAISDYTQTLSKKPEIPTFEVLAFKNRTLGLIDASWSAIRIGIYALHLENWLQYFPLSQILFVSGERLIVDPAGEMAKVQDFLGLKRVVTEKHFYFNKTKGFPCLKKPEDSSAPRCLGKSKGRTHPRIDPDVIHRLRKFYKPFNLMFYQMTGQDFQWEQEEGDK

SEQ ID NO: 2 - protein, 882 aa, Homo sapiens
MPALACLRRLCRHVSPQAVLFLLFIFCLFSVFISAYYLYGWKRGLEPSADAPEPDCGDPPPVAPSRLLPLKPVQAATPSRTDPLVLVFVESLYSQLGQEVVAILESSRFKYRTEIAPGKGDMPTLTDKGRGRFALIIYENILKYVNLDAWNRELLDKYCVAYGVGIIGFFKANENSLLSAQLKGFPLFLHSNLGLKDCSINPKSPLLYVTRPSEVEKGVLPGEDWTVFQSNHSTYEPVLLAKTRSSESIPHLGADAGLHAALHATVVQDLGLHDGIQRVLFGNNLNFWLHKLVFVDAVAFLTGKRLSLPLDRYILVDIDDIFVGKEGTRMKVEDVKALFDTQNELRAHIPNFTFNLGYSGKFFHTGTNAEDAGDDLLLSYVKEFWWFPHMWSHMQPHLFHNQSVLAEQMALNKKFAVEHGIPTDMGYAVAPHHSGVYPVHVQLYEAWKQVWSIRVTSTEEYPHLKPARYRRGFIHNGIMVLPRQTCGLFTHTIFYNEYPGGSSELDKIINGGELFLTVLLNPISIFMTHLSNYGNDRLGLYTFKHLVRFLHSWTNLRLQTLPPVQLAQKYFQIFSEEKDPLWQDPCEDKRHKDIWSKEKTCDRFPKLLIIGPQKTGTTALYLFLGMHPDLSSNYPSSETFEEIQFFNGHNYHKGIDWYMEFFPIPSNTTSDFYFEKSANYFDSEVAPRRAAALLPKAKVLTILINPADRAYSWYQHQRAHDDPVALKYTFHEVITAGSDASSKLRALQNRCLVPGWYATHIERWLSAYHANQILVLDGKLLRTEPAKVMDMVQKFLGVTNTIDYHKTLAFDPKKGFWCQLLEGGKTKCLGKSKGRKYPEMDLDSRAFLKDYYRDHNIELSKLLYKMGQTLPTWLREDLQNTR

SEQ ID NO: 3 - protein, 883 aa, Homo sapiens
MLQLWKVVRPARQLELHRLILLLIAFSLGSMGFLAYYVSTSPKAKEPLPLPLGDCSSGGAAGPGPARPPVPPRPPRPPETARTEPVVLVFVESAYSQLGQEIVAILESSRFRYSTELAPGRGDMPTLTDNTHGRYVLVIYENLLKYVNLDAWSRELLDRYCVEYGVGIIGFFRAHEHSLLSAQLKGFPLFLHSNLGLRDYQVNPSAPLLHLTRPSRLEPGPLPGDDWTIFQSNHSTYEPVLLASLRPAEPAVPGPVLRRARLPTVVQDLGLHDGIQRVLFGHGLSFWLHKLIFVDAVAYLTGKRLCLDLDRYILVDIDDIFVGKEGTRMKVADVEALLTTQNKLRTLVPNFTFNLGFSGKFYHTGTEEEDAGDDMLLKHRKEFWWFPHMWSHMQPHLFHNRSVLADQMRLNKQFALEHGIPTDLGYAVAPHHSGVYPIHTQLYEAWKSVWGIQVTSTEEYPHLRPARYRRGFIHNGIMVLPRQTCGLFTHTIFYNEYPGGSRELDRSIRGGELFLTVLLNPISIFMTHLSNYGNDRLGLYTFESLVRFLQCWTRLRLQTLPPVPLAQKYFELFPQERSPLWQNPCDDKRHKDIWSKEKTCDRLPKFLIVGPQKTGTTAIHFFLSLHPAVTSSFPSPSTFEEIQFFNSPNYHKGIDWYMDFFPVPSNASTDFLFEKSATYFDSEVVPRRGAALLPRAKIITVLTNPADRAYSWYQHQRAHGDPVALNYTFYQVISASSQTPLALRSLQNRCLVPGYYSTHLQRWLTYYPSGQLLIVDGQELRTNPAASMESIQKFLGITPFLNYTRTLRFDDDKGFWCQGLEGGKTRCLGRSKGRRYPDMDTESRLFLTDFFRNHNLELSKLLSRLGQPVPSWLREELQHSSLG

SEQ ID NO: 4 - protein, 262 aa, Homo sapiens
KKLPQALIIGVKKGGTRALLEAIRVHPDVRAVGVEPHFFDRNYEKGLEWYRNVMPKTLDGQITMEKTPSYFVTNEAPKRIHSMAKDIKLIVVVRNPVTRAISDYTQTLSKKPEIPTFEVLAFKNRTLGLIDASWSAIRIGIYALHLENWLQYFPLSQILFVSGERLIVDPAGEMAKVQDFLGLKRVVTEKHFYFNKTKGFPCLKKPEDSSAPRCLGKSKGRTHPRIDPDVIHRLRKFYKPFNLMFYQMTGQDFQWEQEEGDK

SEQ ID NO: 5 - protein, 411 aa, Homo sapiens
MRRRRAGGRTMVERASKFVLVVAGSVCFMLILYQYAGPGLSLGAPGGRAPPDDLDLFPTPDPHYEKKYYFPVRELERSLRFDMKGDDVIVFLHIQKTGGTTFGRHLVQNVRLEVPCDCRPGQKKCTCYRPNRRETWLFSRFSTGWSCGLHADWTELTNCVPGVLDRRDSAALRTPRKFYYITLLRDPVSRYLSEWRHVQRGATWKTSLHMCDGRTPTPEELPPCYEGTDWSGCTLQEFMDCPYNLANNRQVRMLADLSLVGCYNLSFIPEGKRAQLLLESAKKNLRGMAFFGLTEFQRKTQYLFERTFNLKFIRPFMQYNSTRAGGVEVDEDTIRRIEELNDLDMQLYDYAKDLFQQRYQYKRQLERREQRLRSREERLLHRAKEALPREDADEPGRVPTEDYMSHIIEKW

SEQ ID NO: 6 - protein, 645 aa, Homo sapiens
MALPACAVREFEPPRQPERGAPVRTTCPRRHSRVEAELAASRPGSVAASVRAGPPRGVSHGFHTRPLLDKPRKASSSLAGAACAPLFALLSRGRRRRMHVLRRRWDLGSLCRALLTRGLAALGHSLKHVLGAIFSKIFGPMASVGNMDEKSNKLLLALVMLFLFAVIVLQYVCPGTECQLLRLQAFSSPVPDPYRSEDESSARFVPRYNFTRGDLLRKVDFDIKGDDLIVFLHIQKTGGTTFGRHLVRNIQLEQPCECRVGQKKCTCHRPGKRETWLFSRFSTGWSCGLHADWTELTSCVPSVVDGKRDARLRPSRWRIFQILDAASKDKRGSPNTNAGANSPSSTKTRNTSKSGKNFHYITILRDPVSRYLSEWRHVQRGATWKASLHVCDGRPPTSEELPSCYTGDDWSGCPLKEFMDCPYNLANNRQVRMLSDLTLVGCYNLSVMPEKQRNKVLLESAKSNLKHMAFFGLTEFQRKTQYLFEKTFNMNFISPFTQYNTTRASSVEINEEIQKRIEGLNFLDMELYSYAKDLFLQRYQFMRQKEHQEARRKRQEQRKFLKGRLLQTHFQSQGQGQSQNPNQNQSQNPNPNANQNLTQNLMQNLTQSLSQKENRESPKQNSGKEQNDNTSNGTNDYIGSVEKWR

SEQ ID NO: 7 - protein, 471 aa, Homo sapiens
MDERFNKWLLTPVLTLLFVVIMYQYVSPSCTSSCTNFGEQPRAGEAGPPAVPGPARRAQAPPEEWERRPQLPPPPRGPPEGPRGAAAPEEEDEEPGDPREGEEEEEEDEPDPEAPENGSLPRFVPRFNFSLKDLTRFVDFNIKGRDVIVFLHIQKTGGTTFGRHLVKNIRLEQPCSCKAGQKKCTCHRPGKKETWLFSRFSTGWSCGLHADWTELTNCVPAIMEKKDCPRNHSHTRNFYYITMLRDPVSRYLSEWKHVQRGATWKTSLHMCDGRSPTPDELPTCYPGDDWSGVSLREFMDCTYNLANNRQVRMLADLSLVGCYNLTFMNESERNTILLQSAKNNLKNMAFFGLTEFQRKTQFLFERTFNLKFISPFTQFNITRASNVEINEGARQRIEDLNFLDMQLYEYAKDLFQQRYHHTKQLEHQRDRQKRREERRLQREHRDHQWPKEDGAAEGTVTEDYNSQVVRW

SEQ ID NO: 8 - protein, 800 aa, Cricetulus griseus
MAARGRRAWLSMLLGLVLGFVLASRLVLPRASELKRVGPRRRPSPEGCRPGQSASQPGGARGDARGAQLWPHSSAAEGVPRDRNFLFVGVMTAQKYLQTRAVAAYRTWSKTIPGKVEFFSSEGSDTSIPIPIVPLRGVDDSYPPQKKSFMMLKYMHDHYLDKYEWFMRADDDVYIKGDRLESFLRSLNSSEPLFLGQTGLGTTEEMGKLALEPGENFCMGGPGVILSREVLRRMAPHIGKCLREMYTTHEDVEVGRCVRRFAGVQCVWSYEMQQLFYENYEQNKKGYIRDLHSSKIHRAITLHPNKNPPYQYRLHSYMLSRKIAELRHRTIQLHREIVLMSKYSSTEIHKEDLQLGIPPSFMRFQAHQREEILEWEFLTGKYLYSATDGQPPRRGMDSAQREALDDIVMQVMEMINANAKTRGRIIDFKEIQYGYRRVNPMYGAEYILDLLLLYKKHKGKKMTVPVRRHAYLQQTFSKIQFVEHEELDAQELADRINQDSGSLSFLSNSLKKLVPFQLPGSKIEPKEPKEKKINILIPLSGRFDMFVRFMGNFEKTCLIPNQNVKLVVLLFNSDSNPDKAKQVELMRDYRVKYPKADMQVLPVSGGFSRALALEVGSSQFNNESLLFFCDVDLVFTVEFLQRCRANTVLGQQIYFPIIFSQYDPKIVYSGKVPSDNHFAFTQKTGFWRNYGFGITCIFKGDLVRVGGFDVSIQGWGLEDVDLFNKVVQAGLKTFRSQEVGVVHIHHPVFCDPNLDPKQYKMCLGSKASTYGSTQQLAEMWLEKNDPSYSKSGGNGSARTA

SEQ ID NO: 9 - protein, 530 aa, Cricetulus griseus
MVRRGLLGWISRVVILLVLLCCAISVLYMLACTPKGDQEQLGLPWANGPTGKDGYQAVLQEREEQHRNYVNSLKRQIAQLKDELQARSEQLRSGLDQASDATGLRSGWDSAPKAQADLLAFLRGQVDKAEVHAGVKLATEYAAVPFDSFTLQKVYQLETGLTRHPEEKPVRKDKRDELVEAIESALESLNSPVESSPHQRAYTAADFIEGIYRTERDKGTLYELTFKGDHKHEFQRLVLFRPFGPIMKVKEEKLNMANTLINIIVPLARRVDKFRHFMQNFREMCIQQDGRVHLTVVYFGKEEMNEVKGILENTSKAANFRNFTFIQLNGEFSRGKGLDVGARFWKGSNVLLFFCDVDIYFTSEFLNTCRLNTQPGKKVFYPVLFSQYNPGIIYGHQDAVPPLEQQLVIKKETGFWRDFGFGMTCQYQSDFINIGGFDLDIKGWGGEDVHLYRKYLHSNLIVVRTPVRGLFHLWHEKHCMDELTPEQYKMCMQSKAMNEASHGQLGMLVFRHEIEAHLRKQKQKAGSKKT

SEQ ID NO: 10 - protein, 542 aa, Cricetulus griseus
MSRRGPILHSRTQWLLLGLALLFSLVLFMYLLECAPQTDGNASLPGVVRENYGKEYYQALLQEQEEHYQTRATSLKRQIAQLKQELQEMSEKMRSLQEKKNVGANGIGYQGNREQTPSDLLEFLHSQIDRAEVSIGAKLPSEYGVVPFESFTLMKVFQLEMGLTRHPEEKPVRKDKRDELVEVIEAGLEVINNPDEDDEQEDEEGPVGEKLIFNENDFIEGYYRTERDKGTQYELFFKKADLMEYRHVTLFRPFGPLMKVKNEMIDITRSVINIIVPLAERTEAFSQFMQNFRDVCIHQDKRIHLTVVYFGKEGLSKVKSILESITSESDFHNYTLISLNEEFNRGRGLNVGAQAWDKGEVLMFFCDVDIYFSAEFLNSCRLNAEPGKKVFYPVVFSLYNPAIVYANQDVPPPVEQQLVHKKDSGFWRDFGFGMTCQYQSDFLAVGGFDLEVKGWGGEDVHLYRKYLHGDLIVIRTPVPGLFHLWHEKHCADELTPEQYRMCIQSKAMNEASHSHLGMMVFREEIEMHLRKQAYRTNSDTAG

SEQ ID NO: 11 - protein, 873 aa, Homo sapiens
MSFIMKLHRHFQRTVILLATFCMVSIIISAYYLYSGYKQENELSETASEVDCGDLQHLPYQLMEVKAMKLFDASRTDPTVLVFVESQYSSLGQDIIMILESSRFQYHIEIAPGKGDLPVLIDKMKGKYILIIYENILKYINMDSWNRSLLDKYCVEYGVGVIGFHKTSEKSVQSFQLKGFPFSIYGNLAVKDCCINPHSPLIRVTKSSKLEKGSLPGTDWTVFQINHSAYQPVIFAKVKTPENLSPSISKGAFYATIIHDLGLHDGIQRVLFGNNLNFWLHKLIFIDAISFLSGKRLTLSLDRYILVDIDDIFVGKEGTRMNTNDVKALLDTQNLLRAQITNFTFNLGFSGKFYHTGTEEEDEGDDCLLGSVDEFWWFPHMWSHMQPHLFHNESSLVEQMILNKKFALEHGIPTDMGYAVAPHHSGVYPVHVQLYEAWKKVWNIKITSTEEYPHLKPARYRRGFIHKNIMVLPRQTCGLFTHTIFYKEYPGGPKELDKSIQGGELFFTVVLNPISIFMTHLSNYGNDRLGLYTFVNLANFVKSWTNLRLQTLPPVQLAHKYFELFPDQKDPLWQNPCDDKRHRDIWSKEKTCDRLPKFLVIGPQKTGTTALYLFLVMHPSILSNSPSPKTFEEVQFFNRNNYHRGIDWYMDFFPVPSNVTTDFLFEKSANYFHSEEAPKRAASLVPKAKIITILIDPSDRAYSWYQHQRSHEDPAALKFSFYEVISAGPRAPSELRALQKRCLVPGWYASHIERWLVYFPPFQLLIIDGQQLRTDPATVMDEVQKFLGVLPHYNYSEALTFDSHKGFWCQLLEEGKTKCLGKSKGRKYPPMDSDSRTFLSSYYRDHNVELSKLLHKLGQPLPSWLRQELQKVR

SEQ ID NO: 12 - protein, 872 aa, Homo sapiens
MNLIVKLRRSFRTLIVLLATFCLVSIVISAYFLYSGYKQEMTLIETTAEAECTDIKILPYRSMELKTVKPIDTSKTDPTVLLFVESQYSQLGQDIIAILESSRFQYHMVIAPGKGDIPPLTDNGKGKYTLVIYENILKYVSMDSWNRELLEKYCVEYSVSIIGFHKANENSLPSTQLKGFPLNLFNNLALKDCFVNPQSPLLHITKAPKVEKGPLPGEDWTIFQYNHSTYQPVLLTELQTEKSLSSLSSKTLFATVIQDLGLHDGIQRVLFGNNLNFWLHKLIFIDAISFLSGKRLTLSLDRYILVDIDDIFVGKEGTRMNVKDVKALLETQNLLRTQVANFTFNLGFSGKFYHTGTEEEDEGDDLLLRSVDEFWWFPHMWSHMQPHLFHNESSLVEQMILNKEFALEHGIPINMGYAVAPHHSGVYPVHIQLYAAWKKVWGIQVTSTEEYPHLKPARYRKGFIHNSIMVLPRQTCGLFTHTIFYKEYPGGPQELDKSIRGGELFLTILLNPISIFMTHLSNYGNDRLGLYTFVNLVNFVQSWTNLKLQTLPPVQLAHQYFELFPEQKDPLWQNPCDDKRHKDIWSREKTCDHLPKFLVIGPQKTGTTALYLFLLMHPSIISNLPSPKTFEEVQFFNGNNYHKGIDWYMDFFPTPSNTTSDFLFEKSANYFHSEEAPRRAASLVPKAKIITILIDPSDRAYSWYQHQRSHEDPAALRFNFYEVISTGHWAPSDLKTLQRRCLVPGWYAVHIERWLTYFATSQLLIIDGQQLRSDPATVMDEVQKFLGVTPRYNYSEALTFDPQKGFWCQLLEGGKTKCLGKSKGRKYPPMDPESRTFLSNYYRDHNVELSKLLHRLGQPLPSWLRQELQKVR